(19) United States
(12) Patent Application Publication
(10) Pub. No.: US 2025/0120583 A1
(43) Pub. Date: Apr. 17, 2025

(54) DEVICES, SYSTEMS, AND METHODS TO SUPPLY FLUIDS TO AN ENDOSCOPE

(71) Applicant: Boston Scientific Scimed, Inc., Maple Grove, MN (US)

(72) Inventors: Rosangel Aimee Ramos Espinoza, Allston, MA (US); Andrew Usachev, Iowa City, IA (US); Brian Luis, Boylston, MA (US); Ryan V. Wales, Northborough, MA (US)

(73) Assignee: Boston Scientific Scimed, Inc., Maple Grove, MN (US)

(21) Appl. No.: 18/910,872

(22) Filed: Oct. 9, 2024

Related U.S. Application Data
(60) Provisional application No. 63/589,362, filed on Oct. 11, 2023.

(51) Int. Cl. A61B 1/12; A61B 1/00

(52) U.S. Cl. CPC A61B 1/126; A61B 1/00119

(57) ABSTRACT

Methods and systems for providing a flow of fluid to an endoscope. An illustrative container and tube set arranged and configured to couple to an endoscope may comprise a first container configured to contain a fluid, the first container having a first port in fluid communication with a bottom portion thereof, a second container configured to contain a fluid, the second container having a first fluid inlet, wherein a first end of the first fluid inlet is coupled to the first port of the first container and a second end of the first fluid inlet is coupled to the second container, a first water supply tube, and a first gas supply tube. The first water supply tube and the first gas supply tube may each have a lumen in selective fluid communication with the second container and a second end external to the second container.

### CROSS-REFERENCE TO RELATED APPLICATIONS

This application claims the benefit of U.S. Provisional Patent Application Ser. No. 63/589,362 filed on Oct. 11, 2023, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. To enable these capabilities compressed gasses from either the processor or an alternative source are used to increase the pressure within a fluid bottle which either insufflates the working lumen or washes the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day. This may introduce multiple opportunities for contamination to the tube set by either contacting non-sterile surfaces or dropping the tubing on the floor.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a container and tube set arranged and configured to couple to an endoscope for use in an endoscopic procedure may comprise a first container configured to contain a fluid, the first container having a first port in fluid communication with a bottom portion thereof, a second container configured to contain a fluid, the second container having a first fluid inlet including a first end, a second end, and a first lumen, wherein the first end of the first fluid inlet is coupled to the first port of the first container, the second end of the first fluid inlet is coupled to the second container and the first lumen is in selective fluid communication with the first container, a first water supply tube including a first end, a second end, and a second lumen extending therethrough, wherein the second lumen is in selective fluid communication with the bottom portion of the second container and the second end of the first water supply tube is positioned external to the second container, and a first gas supply tube including a first end, a second end, and a third lumen extending therethrough, wherein the third lumen is in operative fluid communication with the second container and the second end of the first gas supply tube is positioned external to the second container.

Alternatively or additionally to any of the examples above, in another example, the container and tube set may further comprise a second water supply tube including a first end, a second end, and a fourth lumen extending therethrough, wherein the fourth lumen is in selective fluid communication with the bottom portion of the second container and the second end of the second water supply tube is positioned external to the second container.

Alternatively or additionally to any of the examples above, in another example, the container and tube set may further comprise a second water supply tube including a first end, a second end, and a fourth lumen extending therethrough, wherein the fourth lumen is in selective fluid communication with the bottom portion of the first container and the second end of the second water supply tube is positioned external to the first container.

Alternatively or additionally to any of the examples above, in another example, the second container may further comprise a partition extending from the bottom portion towards the top portion.

Alternatively or additionally to any of the examples above, in another example, the partition may divide the second container into a first chamber and a second chamber.

Alternatively or additionally to any of the examples above, in another example, the partition may terminate a distance from the top portion of the second container.

Alternatively or additionally to any of the examples above, in another example, the partition may be laterally disposed between a second port of the second container and a third port of the second container.

Alternatively or additionally to any of the examples above, in another example, the first end of the first water supply tube may be coupled to the second port of the second container and the first end of the first gas supply tube may be coupled to the third port of the second container.

Alternatively or additionally to any of the examples above, in another example, the partition may extend to the top portion of the second container.

Alternatively or additionally to any of the examples above, in another example, the container and tube set may further comprise comprising a one-way valve disposed within the partition.

Alternatively or additionally to any of the examples above, in another example, the container and tube set may further comprise a third container configured to contain a fluid, the third container having a second port in fluid communication with a bottom portion thereof.

Alternatively or additionally to any of the examples above, in another example, the container and tube set may further comprise a second water supply tube including a first end, a second end, and a fourth lumen extending therethrough, wherein the fourth lumen is in selective fluid communication with the bottom portion of the third container and the second end of the second water supply tube is positioned external to the third container.

Alternatively or additionally to any of the examples above, in another example, the first container may comprise a collapsible bag.

Alternatively or additionally to any of the examples above, in another example, the second container may comprise a collapsible bag.

Alternatively or additionally to any of the examples above, in another example, the container and tube set may further comprise a one-way valve positioned between the first end and the second end of the first fluid inlet.

In another example, a container and tube set arranged and configured to couple to an endoscope for use in an endoscopic procedure may comprise a first container configured to contain a fluid, the first container having a first port in fluid communication with a top portion thereof, a first gas supply tube including a first end, a second end, and a first lumen extending therethrough, wherein the first lumen is in operative fluid communication with the first container and the second end of the first gas supply tube is positioned external to the first container, a first water supply tube including a first end, a second end, and a second lumen extending therethrough, wherein the second lumen is in selective fluid communication with the fluid of the first container and the second end of the first water supply tube is positioned external to the second container, and a three-way port coupled the second end of the first water supply tube.

Alternatively or additionally to any of the examples above, in another example, the container and tube set may further comprise a second water supply tube including a first end, a second end, and a third lumen extending therethrough, wherein the first end is coupled to the three-way port, the third lumen is in selective fluid communication with the first water supply tube and the second end of the second water supply tube is positioned external to the first container.

Alternatively or additionally to any of the examples above, in another example, the container and tube set may further comprise a third water supply tube including a first end, a second end, and a fourth lumen extending therethrough, wherein the first end is coupled to the three-way port, the fourth lumen is in selective fluid communication with the first water supply tube and the second end of the third water supply tube is positioned external to the first container.

Alternatively or additionally to any of the examples above, in another example, the first port may be configured to be selectively fluidly coupled with a fluid source.

In another example, a container and tube set arranged and configured to couple to an endoscope for use in an endoscopic procedure may comprise a first container configured to contain a fluid, the first container having a first port in fluid communication with a top portion thereof, a partition extending from a top portion of the first container to a bottom portion of the first container, the partition dividing the first container into a first chamber and a second chamber, a one-way valve positioned in the partition, the one-way valve configured to allow a flow of fluid from the first chamber to the second chamber, a first water supply tube including a first end, a second end, and a first lumen extending therethrough, wherein the first lumen is in selective fluid communication with first chamber of the first container and the second end of the first water supply tube is positioned external to the first container, a second water supply tube including a first end, a second end, and a second lumen extending therethrough, wherein the second lumen is in selective fluid communication with the bottom portion of the second chamber and the second end of the first water supply tube is positioned external to the first container, and a first gas supply tube including a first end, a second end, and a third lumen extending therethrough, wherein the third lumen is in operative fluid communication with the second chamber and the second end of the first gas supply tube is positioned external to the first container.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3 depicts another illustrative endoscope system having an alternative fluid supply system;

FIG. 4 depicts another illustrative endoscope system having an alternative fluid supply system;

FIG. 5 depicts another illustrative endoscope system having an alternative fluid supply system;

FIG. 6 depicts another illustrative endoscope system having an alternative fluid supply system;

FIG. 7 depicts an illustrative container and tube set for use with an endoscope system; and

FIG. 8 depicts another illustrative endoscope system having an alternative fluid supply system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. To enable these capabilities compressed gasses from either the processor or an alternative source are used to increase the pressure within a fluid bottle which either insufflates the working lumen or washes the lens of the endoscope. Additionally, a peristaltic pump can be used to irrigate the working lumen of debris. One of the challenges faced during endoscopic procedures is that the common water bottle and tube set used contain a maximum of 1 liter of water and are not designed to be refilled. This may force nurses/technicians to replace the water bottle multiple times a day which may introduce multiple opportunities for contamination to the tube set by either contacting non-sterile surfaces or dropping the tubing on the floor. Additionally, current water bottle and tube sets may leak if not threaded properly. Finally, the water bottle may require a level surface to be placed properly which, in an endoscopy suite, may be at a premium. Disclosed herein are methods and systems to reduce or eliminate the need to disconnect the tube set and use a second bottle.

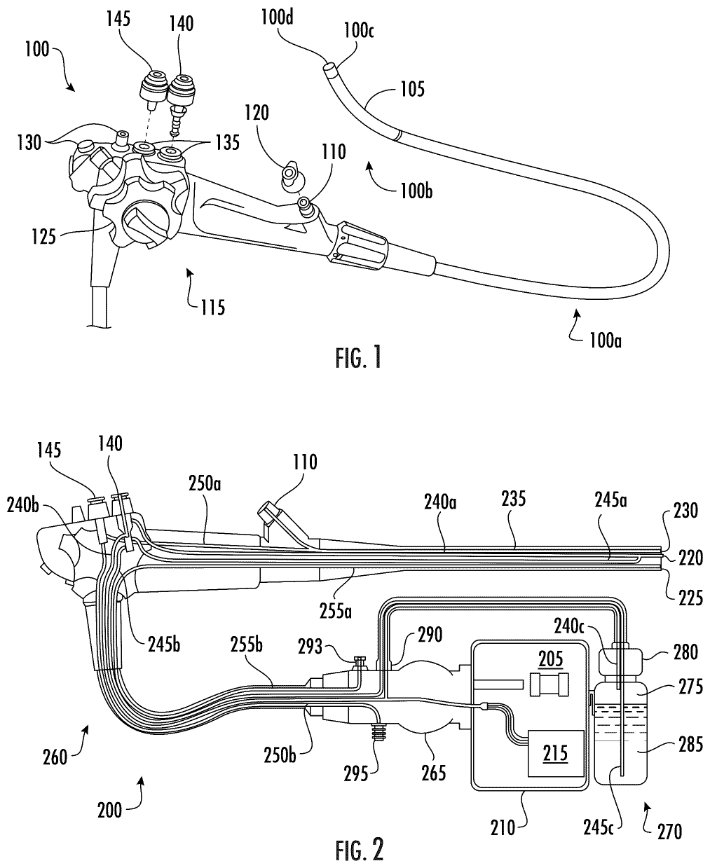

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top of the water reservoir. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

It is contemplated that other arrangements for the fluid sources may be used as desired. For example, in some cases, water for irrigation and lens wash may come from a same container. Some illustrative systems and method to supply fluids to the endoscope are described in commonly assigned U.S. Patent Application No. 63/419,900, titled DEVICES, SYSTEMS, AND METHODS TO SUPPLY FLUIDS TO AN ENDOSCOPE, the disclosure of which is hereby incorporated by reference.

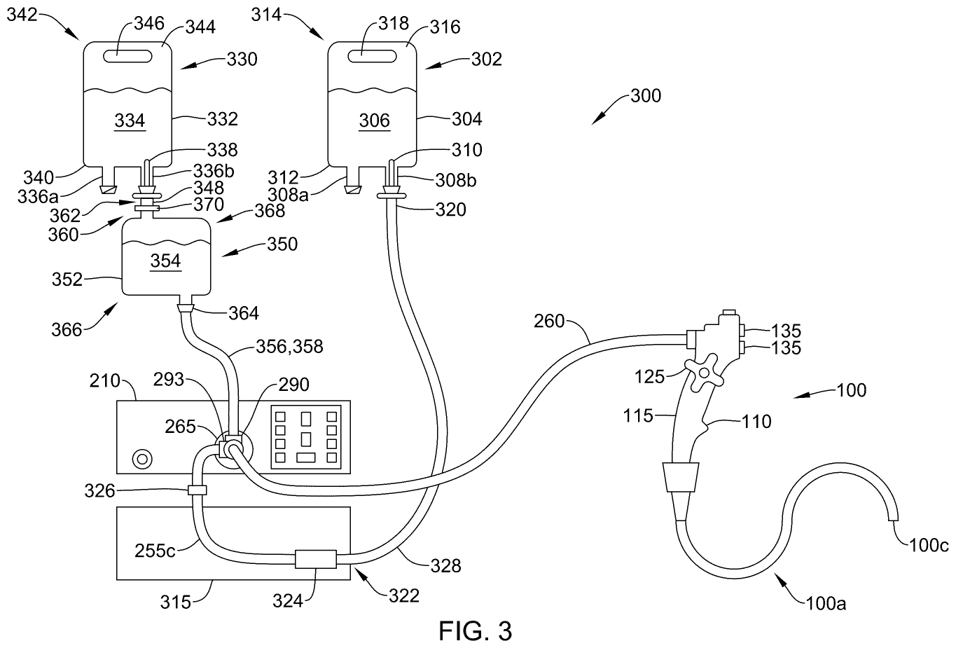

As described above, it may be desirable to reduce opportunities for contamination to the tube set 240c, 245c during replacement of the water reservoir(s). FIG. 3 depicts a schematic view of an illustrative endoscopic system 300 which may reduce the number of water reservoir changes and/or reduce opportunities for contamination during replacement of the water reservoir(s). Further, the system 300 may leverage existing pathways and resources available in the endoscopy suit. In some cases, the system 300 may reduce a length of the irrigation supply tubing 255c, the gas supply tubing 240c, and/or the lens wash supply tubing 245c. The system 300 may include components similar to the endoscope and endoscope systems described with regard to FIGS. 1-2; however, not all features may be described or shown here.

The system 300 may include a first fluid reservoir 302 including a first container 304 configured to hold a fluid 306. In the illustrated embodiment, the first container 304 is fluidly coupled to the upstream irrigation tubing 328 and is configured to provide fluid for irrigation to the endoscope 100. Generally, the irrigation tubing 328 may be a water or fluid supply line or tube for supplying water or other fluid to an endoscope 100. While not explicitly shown, the first reservoir 302 may include printed lines, numbers, or other visual indicia to allow a user to easily determine how much fluid is left in the first reservoir 302.

The first container 304 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first container 304 may be entirely translucent, entirely opaque, or combinations thereof. In some cases, the first container 304 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first container 304 may be variable. For example, the volume of the first container 304 may be 500 milliliters (mL) or greater, 1000 mL or greater, 2000 mL or greater, 3000 mL, 4000 mL or greater, etc. The volume may be less than 500 mL or greater than 4000 mL, as desired. The first reservoir 302 may be pre-filled (e.g., prior to entering the procedure suite or at the time of manufacturing) with water or other fluid. In some cases, the clinician may select the first reservoir 302 from a plurality of differently sized available reservoirs based on the number and/or types of procedures expected for a day. By selecting a first reservoir 302 having a volume large enough to accommodate an entire day of procedures, the need for replacing the sterile fluid source (e.g., the first reservoir 302) may be reduced or eliminated. It is contemplated that flexible bags may utilize less plastic (or other material) than a bottle designed to hold a similar amount of fluid. Thus, the use of a flexible bag as a fluid first reservoir 302 may increase the level of environmental sustainability of the system 300. For example, if the user sets up the system with a 3000 mL (3 liter) bag first reservoir 302 and therefore does not need to utilize three individual one liter bottles, a significant reduction of waste may be realized. It is further contemplated that when disposed of or discarded, a bag first reservoir 302 may occupy less volume than a bottle capable of holding an equivalent amount of fluid.

The first reservoir 302 may further include one or more ports 308a, 308b, such as, but not limited to a spike port or a septum port, extending from and in selective fluid communication with an interior of the first container 304. The ports 308a, 308b may be formed as a monolithic structure with the first container 304. The ports 308a, 308b may be generally tubular structures with each port 308a, 308b defining a lumen extending therethrough. The lumens of the ports 308a, 308b may be configured to selectively fluidly couple the interior of the first container 304 with another component, such as, but not limited to, a water or fluid supply tube. In some embodiments, the ports 308a, 308b may be positioned adjacent to a bottom end 312 of the first reservoir 302. However, this is not required. The ports 308a, 308b may be positioned in other locations, as desired. If the ports 308a, 308b are positioned at a location other than the bottom end 312 of the first container 304, a dip tube or tube extension may be required to access the fluid at the bottom of the first container 304. In some cases, at least one port 308b may be configured to be coupled to the upstream irrigation tubing (or water/fluid supply tube) 328 while the other port 308a may be configured to allow the user to add additives to the fluid 306 (e.g., irrigation fluid). While the first reservoir 302 is illustrated as including two ports 308a, 308b, the first reservoir 302 may include one port or more than two ports, as desired.

While not explicitly shown, the ports 308a, 308b may each include a removable cap or seal configured to form a fluid tight seal with the port 308a, 308b. The removable cap or seal may help to maintain the sterility of the ports 308a, 308b. The removable cap or seal may be coupled to a free end of the ports 308a, 308b using a number of different techniques. For example, the cap or seal may be coupled to the port 308a, 308b using a threaded engagement, a friction fit, a snap fit, etc. In other instances, the cap or seal may be removed through a twisting motion configured to break the cap or seal from the port 308a, 308b. Once the cap or seal has been removed, the port 308a, 308b may be pierced with a spike tip or spike port adaptor 310 that is coupled to the upstream irrigation tubing 328. For example, in addition to the removable cap or seal, the port 308a, 308b may include an internal seal disposed within a lumen of the port 308a, 308b that may be punctured or pierced by the spike port adaptor 310. The internal seal may be configured to prevent fluid 306 from leaking from the first container 304 prior to the spike port adaptor 310 being inserted into the port 308a, 308b. In some embodiments, the internal seal may be self-sealing such that upon removal of the spike port adaptor 310 fluid is prevented from leaking from the port 308a, 308b. The outer surface of the spike port adaptor 310 may form an interference fit with the inner surface of the port 308a, 308b. The fit and/or coupling between the spike port adaptor 310 and the port 308a, 308b may be sufficient to remain in place when the irrigation supply tube 328 and/or other tubing sets are coupled to the spike port adaptor 310. It is contemplated that the spike port adaptor 310 may be inserted into one of the ports 308a, 308b utilizing universally used aseptic techniques such as those used with IV fluid bags. This may help reduce infection risk by maintaining sterile components, not introducing contaminants into the fluid 306, etc. It is further contemplated that additives may be added to the fluid 306 using similar aseptic techniques via one of the ports 308a, 308b.

The first reservoir 302 may include a handle 316 positioned adjacent to a top portion 314 thereof. The handle 316 may define an opening or through hole 318 for receiving a hand or hook therethrough to carry the first reservoir 302. In some cases, the handle 316 may include an undulating surface configured to provide a more ergonomic grip for the user. It is contemplated that the handle 316 may be formed from a similar material as the first container 304 or a different material, as desired. In some examples, the handle 316 may be formed from polyethylene terephthalate (PET), polypropylene (PP), etc. The handle 316 may allow the first reservoir 302 to be hung from a hook, such as, but not limited to an IV stand. Hanging the first reservoir 302 may allow the first reservoir 302 to be positioned above the level of an endoscope cart which may enable the user to see the fluid 306 level at any time. This may help the clinician avoid running out of fluid during a procedure. Additionally, elevating the reservoir may eliminate the need for the clinician to bend or stoop during setup of the system 300 and/or to change the first reservoir 302. In some cases, head pressure generated from elevating the first reservoir 302 may enable rapid priming of the irrigation circuit (and/or lens wash circuit if so connected) which may save time during setup. It is further contemplated that hanging the first reservoir 302 from a hook or IV stand may allow the first reservoir 302 to be positioned away from expensive capital equipment thus reducing or eliminating the potential for fluid running or flowing inadvertently onto the capital equipment and causing damage or destruction.

The first reservoir 302 may be connected in fluid communication with a lumen of an upstream irrigation supply tube 328. The upstream irrigation supply tube 328 extends from a second end region 322 external to the first container 304 and positioned a pump head 324 of the peristaltic irrigation pump 315 to a first end 320. The first end 320 is coupled to the spike port adaptor 310 which in turn is configured to extend through a lumen of the port 308b and pierce a seal within the lumen of the port 308b to fluidly couple the interior of the first container 304 with the lumen of the upstream irrigation supply tube 328. The second end of the upstream irrigation supply tube 328 is configured to be fluidly coupled with an irrigation lumen of the endoscope 100. When irrigation water is required, fluid is pumped from the first container 304 by operating the irrigation pump 315, such as by depressing a footswitch (not shown), and flows from the first reservoir 302, through the upstream irrigation supply tubing 328 and a branched connector 350, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in-line with the downstream irrigation supply tubing 255c. The flow control valve 326 may prevent the unintentional flow of fluid from the first container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 302 and the irrigation pump 315. The flow control valve 326 may also prevent fluid from leaking from the downstream irrigation supply tube 255c when the endoscope 100 is changed between patients.

If there is a need to replace the first reservoir 302 with a new full bag, for example when the first reservoir 302 is empty or near empty, the user may hang the new bag near the first reservoir 302 to be replaced. The user may then disengage the spike port adaptor 310 from the port 308b and insert the spike port adaptor 310 into a port of the new bag. This may be performed without requiring the clinician to bend or stoop to access the first reservoir 302. The port 308b may self-seal to prevent fluid leaks from the first reservoir 302 as the first reservoir 302 is being replaced. This method of replacing the first reservoir 302 may have a lower risk of introducing contaminants into the systems relative to traditional bottle systems. For example, the change out method described herein may allow the first reservoir 302 to be changed out without having tubing dangling from a cap (as in a bottle system). Additionally, the change out method described herein may allow the first reservoir 302 to be changed out independently from the second reservoir 330 which may further reduce the risk of contamination to the tubing. Further, the system 300 may remain largely closed as the first reservoir 302 is changed out.

The system 300 may include a second fluid reservoir 330 including a second container 332 configured to hold a second volume of fluid 334. In the illustrated embodiment, the second container 332 is fluidly coupled to a third fluid reservoir 350 via a fluid supply tube 348. Generally, the fluid supply tube 348 may be a water or fluid supply line or tube for supplying water or other fluid to the third reservoir 350 which in turn supplies water or other fluid to an endoscope. The third reservoir 350 may include a third container 352 configured to hold a third volume of fluid 354. In the illustrated embodiment, the third container 352 is fluidly coupled to the gas and lens wash supply tubing 356, 358 and is configured to provide fluid for lens wash to the endoscope 100. Generally, the lens wash supply tubing 358 may be a water or fluid supply line or tube for supplying water or other fluid to an endoscope. While not explicitly shown, the second reservoir 330 and/or the third reservoir 350 may include printed lines, numbers, or other visual indicia to allow a user to easily determine how much fluid is left in the second reservoir 330 and/or the third reservoir 350.

The second container 332 and/or the third container 352 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the second container 332 and/or the third container 352 may be entirely translucent, entirely opaque, or combinations thereof. In some cases, the second container 332 and/or the third container 352 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the second container 332 and/or the third container 352 may be variable. For example, the volume of the second container 332 and/or the third container 352 may be 500 milliliters (mL) or greater, 1000 mL or greater, 2000 mL or greater, 3000 mL, 4000 mL or greater, etc. The volume may be less than 500 mL or greater than 4000 mL, as desired. The second reservoir 330 may be pre-filled (e.g., prior to entering the procedure suite or at the time of manufacturing) with water or other fluid. In some cases, the clinician may select the second reservoir 330 from a plurality of differently sized available reservoirs based on the number and/or types of procedures expected for a day. In the illustrated embodiments, the second reservoir 330 may supply fluid to the third reservoir 350. By selecting a second reservoir 330 having a volume large enough to accommodate an entire day of procedures, the need for replacing the sterile fluid source (e.g., the second reservoir 330) may be reduced or eliminated. In some cases, the second reservoir 330 may be used to periodically refill the third reservoir 350. Thus, the volume of the second reservoir 330 may be greater than the volume of the third reservoir 350, although this is not required. It is further contemplated that, in some embodiments, one or both of the second or third reservoirs 330, 350 may be a rigid bottle.

The second reservoir 330 may further include one or more ports 336a, 336b, such as, but not limited to a spike port or a septum port, extending from and in selective fluid communication with an interior of the second container 332. The ports 336a, 336b may be formed as a monolithic structure with the second container 332. The ports 336a, 336b may be generally tubular structures with each port 336a, 336b defining a lumen extending therethrough. The lumens of the ports 336a, 336b may be configured to selectively fluidly couple the interior of the second container 332 with another component, such as, but not limited to, the third container 352. In some embodiments, the ports 336a, 336b may be positioned adjacent to a bottom end 340 of the second reservoir 330. However, this is not required. The ports 336a, 336b may be positioned in other locations, as desired. If the ports 336a, 336b are positioned at a location other than the bottom end 340 of the second container 332, a dip tube or tube extension may be required to access the fluid at the bottom of the second container 332. In some cases, at least one port 336b may be configured to be coupled to the fluid supply tube 348 while the other port 336a may be configured to allow the user to add additives to the fluid 334 (e.g., irrigation fluid). While the second reservoir 330 is illustrated as including two ports 336a, 336b, the second reservoir 330 may include one port or more than two ports, as desired.

While not explicitly shown, the ports 336a, 336b may each include a removable cap or seal configured to form a fluid tight seal with the port 336a, 336b. The removable cap or seal may help to maintain the sterility of the ports 336a, 336b. The removable cap or seal may be coupled to a free end of the ports 336a, 336b using a number of different techniques. For example, the cap or seal may be coupled to the port 336a, 336b using a threaded engagement, a friction fit, a snap fit, etc. In other instances, the cap or seal may be removed through a twisting motion configured to break the cap or seal from the port 336a, 336b. Once the cap or seal has been removed, the port 336a, 336b may be pierced with a spike tip or spike port adaptor 338 that is coupled to the fluid supply tube 348. For example, in addition to the removable cap or seal, the port 336a, 336b may include an internal seal disposed within a lumen of the port 336a, 336b that may be punctured or pierced by the spike port adaptor 338. The internal seal may be configured to prevent fluid 334 from leaking from the second container 332 prior to the spike port adaptor 338 being inserted into the port 336a, 336b. In some embodiments, the internal seal may be self-sealing such that upon removal of the spike port adaptor 338 fluid is prevented from leaking from the port 336a, 336b. The outer surface of the spike port adaptor 338 may form an interference fit with the inner surface of the port 336a, 336b. The fit and/or coupling between the spike port adaptor 338 and the port 336a, 336b may be sufficient to remain in place when the fluid supply tube 348 and/or other tubing sets are coupled to the spike port adaptor 338. It is contemplated that the spike port adaptor 338 may be inserted into one of the ports 336a, 336b utilizing universally used aseptic techniques such as those used with IV fluid bags. This may help reduce infection risk by maintaining sterile components, not introducing contaminants into the fluid 334, etc.

The second reservoir 330 may include a handle 344 positioned adjacent to a top portion 342 thereof. The handle 344 may define an opening or through hole 346 for receiving a hand or hook therethrough to carry the second reservoir 330. In some cases, the handle 344 may include an undulating surface configured to provide a more ergonomic grip for the user. It is contemplated that the handle 344 may be formed from a similar material as the second container 332 or a different material, as desired. In some examples, the handle 344 may be formed from polyethylene terephthalate (PET), polypropylene (PP), etc. The handle 344 may allow the second reservoir 330 to be hung from a hook, such as, but not limited to an IV stand. Hanging the second reservoir 330 may allow the second reservoir 330 to be positioned above the level of an endoscope cart which may enable the user to see the fluid 334 level at any time. This may help the clinician avoid running out of fluid during a procedure. Additionally, elevating the reservoir may eliminate the need for the clinician to bend or stoop during setup of the system 300 and/or to change the second reservoir 330. In some cases, head pressure generated from the elevating the second reservoir 330 may enable rapid priming of the lens wash circuit which may save time during setup. It is further contemplated that hanging the second reservoir 330 from a hook or IV stand may allow the second reservoir 330 to be positioned away from expensive capital equipment thus reducing or eliminating the potential for fluid running or flowing inadvertently onto the capital equipment and causing damage or destruction.

The second reservoir 330 may be connected in fluid communication with a lumen of fluid supply tube 348. The fluid supply tube 348 extends from a second end region 360 external to the second container 332 and coupled to and in fluid communication with the third container 352 to a first end 362. The first end 362 is coupled to the spike port adaptor 338 which in turn is configured to extend through a lumen of the port 336b and pierce a seal within the lumen of the port 336b to fluidly couple the interior of the second container 332 with the lumen of the fluid supply tube 348.

In the illustrated embodiment, the third container 352 is fluidly coupled to the gas and lens wash supply tubing 356, 358 and is configured to provide fluid for lens wash to the endoscope 100. Generally, the lens wash supply tubing 358 may be a water or fluid supply line or tube for supplying water or other fluid to an endoscope. The gas and lens wash supply tubing 356, 358 may be coaxially arranged. For example, the gas supply tubing 356 may define a lumen that is sufficiently large in diameter to encompass a smaller diameter lens wash tubing 358, coaxially received within the gas supply tubing 356, as well as provide air to the water source in an annular space surrounding the lens wash tubing 358 to pressurize the third reservoir 350. The lens wash supply tubing 358 may be configured to exit the lumen defined by the coaxial gas supply tubing 356 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 356, 358 may be arranged in a side-by-side arrangement.

The third reservoir 350 may further include one or more ports 364, such as, but not limited to a spike port or a septum port, extending from and in selective fluid communication with an interior of the third container 352. The port 364 may be formed as a monolithic structure with the third container 352. The port 364 may be a generally tubular structure with the port 364 defining a lumen extending therethrough. The lumen of the port 364 may be configured to selectively fluidly couple the interior of the third container 352 with another component, such as, but not limited to, fluid/water/gas supply tube(s). In some cases, the port 364 may be configured to be coupled to the gas and lens wash supply tubing 356, 358. In some embodiments, the port 364 may be positioned adjacent to a bottom end 366 of the third reservoir 350. However, this is not required. The port 364 may be positioned in other locations, as desired. If the port 364 is positioned at a location other than the bottom end 366 of the third container 352, a dip tube or tube extension may be required (e.g., coupled to the lens wash supply tubing 358) to access the fluid at the bottom of the third container 352. While the third reservoir 350 is illustrated as including one port 364, the third reservoir 350 may include more than one port, as desired.

While not explicitly shown, the port 364 may include a removable cap or seal configured to form a fluid tight seal with the port 364. The removable cap or seal may help to maintain the sterility of the port 364. The removable cap or seal may be coupled to a free end of the port 364 using a number of different techniques. For example, the cap or seal may be coupled to the port 364 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 364. Once the cap or seal has been removed, the port 364 may be pierced with a spike tip or spike port adaptor (not explicitly shown) that is coupled to the gas and lens wash supply tubing 356, 358. For example, in addition to the removable cap or seal, the port 364 may include an internal seal disposed within a lumen of the port 364 that may be punctured or pierced by the spike port adaptor. The internal seal may be configured to prevent fluid 354 from leaking from the third container 352 prior to the spike port adaptor being inserted into the port 364. In some embodiments, the internal seal may be self-sealing such that upon removal of the spike port adaptor fluid is prevented from leaking from the port 364. The outer surface of the spike port adaptor may form an interference fit with the inner surface of the port 364. The fit and/or coupling between the spike port adaptor and the port 364 may be sufficient to remain in place when the gas and fluid supply tubing 356, 358 and/or other tubing sets are coupled to the spike port adaptor. It is contemplated that the spike port adaptor may be inserted into the port 364 utilizing universally used aseptic techniques such as those used with IV fluid bags. This may help reduce infection risk by maintaining sterile components, not introducing contaminants into the fluid 354, etc. It is further contemplated that additives may be added to the fluid 354 using similar aseptic techniques via one of the ports 364, if so desired. In some cases, other coupling mechanisms may be used as desired to couple the gas and lens wash supply tubing 356, 358 to the port 364. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 356 extends from a second end external to the third container 352 to the port 364. While not explicitly shown, the gas supply tubing 356 may extend into the interior of the third container 352 and terminate within a reservoir gap (e.g., above the level of the fluid 354). However, in some cases, the gas supply tubing 356 may terminate within the fluid 354. A lumen extends through the gas supply tubing 356 for receiving a flow of air and/or gas therethrough. The lumen of the gas supply tubing 356 may be in operative fluid communication with a top portion of the interior of the third container 352. The lens wash supply tubing 358 extends from a second end external to the third reservoir 350 to a first end in fluid communication with a bottom portion 366 of the third container 352. In some embodiments, the lens wash supply tubing 358 may terminate at the port 364. A lumen extends through the lens wash supply tubing 358 for receiving a flow of fluid therethrough. The lumen of the lens wash supply 358 is in selective operative fluid communication with a bottom portion 366 of the third container 352. In the illustrated embodiment, the gas supply tubing 356 and the lens wash supply tubing 358 may couple to the third container 352 through a single or common opening (e.g., port 364). For example, the gas supply tubing 356 and the lens wash supply tubing 358 may be coaxially arranged. However, this is not required. In some cases, the gas supply tubing 356 and the lens wash supply tubing 358 may extend in a side by side arrangement or may be separately connected to the third container 352 in different locations.

The third container 352 may further include the fluid supply tube 348. While the fluid supply tube 348 is illustrated as being adjacent to or extending from a top portion 368 of the third container 352, the fluid supply tube 348 may be positioned at other locations about the third container 352, as desired. In some embodiments, the fluid supply tube 348 may be a tubular member formed as a single monolithic structure with the third container 352. In other embodiments, the fluid supply tube 348 may include tubular components releasably coupled to ports (similar in form and function to port 364) formed in or with the third container 352.

The fluid supply tube 348 may be in selective fluid communication with the second reservoir 330. For example, as described above, the first end 362 of the fluid supply tube 348 may be coupled to the spike port adaptor 338 which in turn is coupled to the second port 336b of the second reservoir 330. A flow control mechanism, such as, but not limited to, a one-way valve 370 may be positioned between the first end 362 and the second end 360 of the fluid supply tube 348 to selectively fluidly couple the third container 352 with the second container 332. The one-way valve 370 may be configured to be opened to allow fluid to selectively pass from the second reservoir 330 to the third reservoir 350 while preventing fluid (e.g., gas, water, or other fluid) from exiting the third container 352 and entering the second container 332. In some embodiments, the one-way valve 370 may be replaced with a clamp which may compress the fluid supply tube 348 to selectively fluidly isolate the third container 352 from the second container 332 and removed to selectively couple the third container 352 with the second container 332. In yet other embodiments, the one-way valve 370 may be replaced with a spring-loaded valve, a stopcock, or other two-way valve. When it is desired to add fluid to the third reservoir 350 from the second reservoir 330, the one-way valve 370 (or other flow control mechanism) may be opened or released. Fluid may then flow from the second reservoir 330 to the third reservoir 350.

While not explicitly shown, the third container 352 may include a second fluid supply tube which may be an alternative gas supply tubing configured to be coupled to an alternative gas supply (e.g., CO₂ hospital house gas source). The second fluid supply tube may extend from a second end external to the third container 352 to a first end coupled to the third container 352. The alternative gas supply may be used to pressurize the third container 352 to supply lens wash to the endoscope 100 and/or to provide insufflation. A lumen extends through the second fluid supply tube for receiving a flow of gas therethrough. The lumen of the second fluid supply tube is in operative fluid communication with a top portion of the third container 352. The flow of the CO₂ through the system 300 may be similar to that described above. For example, in the neutral state, CO₂ gas flows through the second fluid supply tube into the third container 352, up the gas supply tubing 356 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid supply tube into the third container 352, up the gas supply tubing 356 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the third reservoir 350 is maintained by delivering gas through the second fluid supply tube. It is contemplated that the one-way valve 370 is in the closed configuration during delivery of the CO₂ gas to allow the container 352 to pressurize. In some instances, the one-way valve 370 may be configured to close without user intervention in response to the delivery of CO₂ to the third container 352. In some embodiments, the system 300 may include a branched connector (such as, but not limited to a “Y” or “T” connector) at the second fluid supply tube to allow either air or CO₂ to be used for pressurization or insufflation. Alternatively, air may be supplied to the third reservoir 350 via the gas supply tube 356 from a pressurizing pump 215 in the processing unit 210. It is further contemplated that the second fluid supply tube may include a pressure relief valve, such as, but not limited to, a 3-way stopcock, a clamp, a spring-loaded valve, or the like to vent pressure within the third container 352 and/or to block a flow of pressurized gas to the third container 352 during refilling of the third container 352, during procedure change-overs, and/or during equipment change-overs.

As the pressurized third container 352 is fluidly isolated from the second container 332 when the one-way valve 370 is closed, it is contemplated that the clinician may replace the second reservoir 330 with a new (e.g., full) reservoir without losing patient insufflation. Loss of patient insufflation may result in a loss of position of the endoscope 100 within the body. In current one or two bottle systems, it may not be possible to replace the water reservoir(s) without loss of patient insufflation.

If there is a need to replace the second reservoir 330 with a new full bag, for example when the second reservoir 330 is empty or near empty, the user may hang the new bag near the second reservoir 330 to be replaced. The user may then disengage the spike port adaptor 338 from the port 336b and insert the spike port adaptor 338 into a port of the new bag. This may be performed without requiring the clinician to bend or stoop to access the second reservoir 330. The port 336b may self-seal to prevent fluid leaks from the second reservoir 330 as the second reservoir 330 is being replaced. This method of replacing the second reservoir 330 may have a lower risk of introducing contaminants into the systems relative to traditional bottle systems. For example, the change out method described herein may allow the second reservoir 330 to be changed out without having tubing dangling from a cap (as in a bottle system). Additionally, the change out method described herein may allow the second reservoir 330 to be changed out independently from the first reservoir 302 which may further reduce the risk of contamination to the tubing. Further, the system 300 may remain largely closed as the second reservoir 330 is changed out.

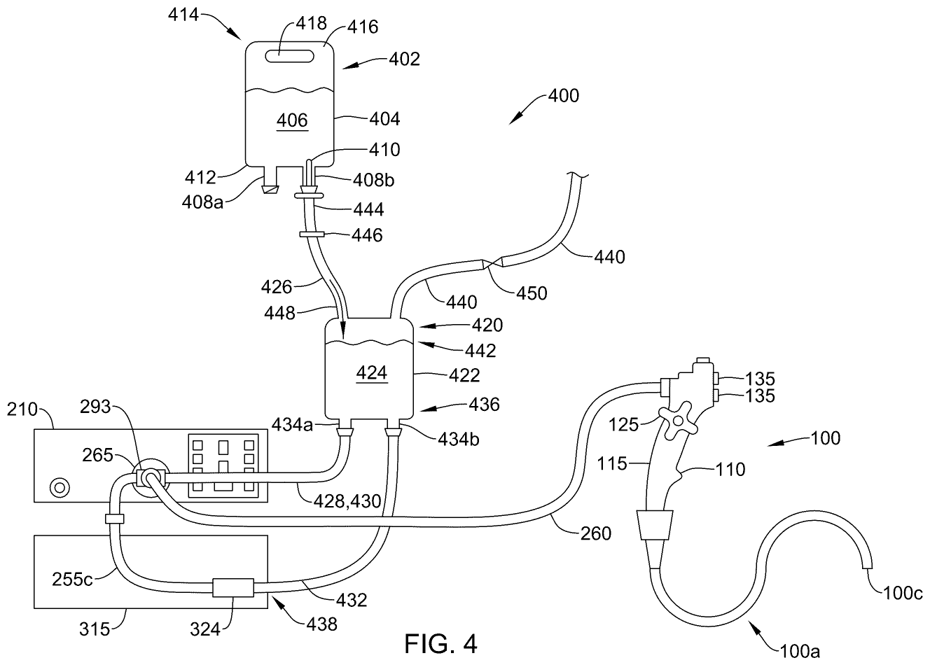

FIG. 4 depicts a schematic view of another illustrative endoscopic system 400 which may reduce the number of water reservoir changes and/or reduce opportunities for contamination during replacement of the water reservoir(s). The system 400 may include a number of advantages over the current bottle system described above. The system 400 may include components similar to the endoscope and endoscope systems described with regard to FIGS. 1-2; however, not all features may be described or shown here.

Generally, the system 400 may include a first reservoir 402 and a second reservoir 420. The first reservoir 402 may be configured to supply water or fluid for both irrigation (e.g., via the second reservoir 420) and lens wash (e.g., via the second reservoir 420). This may allow a single fluid source to be used to provide fluid for both irrigation and lens wash. While not explicitly shown, the reservoirs 402, 420 may include printed lines, numbers, or other visual indicia to allow a user to easily determine how much fluid is left in the reservoirs 402, 420.

The first reservoir 402 may include a first container 404 configured to hold a first volume of fluid 406. In the illustrated embodiment, the first container 404 may be selectively fluidly coupled to a first fluid inlet 426 of the second reservoir 420. Generally, the first fluid inlet 426 may be a water or fluid supply line or tube for supplying water or other fluid to the second reservoir 420. The second reservoir 420 may include a second container 422 configured to hold a second volume of fluid 424. In the illustrated embodiment, the second container 422 is fluidly coupled to the gas and lens wash supply tubing 428, 430 and is configured to provide fluid for lens wash to the endoscope 100. Generally, the lens wash supply tubing 430 may be a water or fluid supply line or tube for supplying water or other fluid to an endoscope. The gas and lens wash supply tubing 428, 430 may be coaxially arranged. For example, the gas supply tubing 428 may define a lumen that is sufficiently large in diameter to encompass a smaller diameter lens wash tubing 430, coaxially received within the gas supply tubing 428, as well as provide air to the water source in an annular space surrounding the lens wash tubing 430 to pressurize the second reservoir 420. The lens wash supply tubing 430 may be configured to exit the lumen defined by the coaxial gas supply tubing 428 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 428, 430 may be arranged in a side-by-side arrangement. Further, the second container 422 is fluidly coupled to the irrigation supply tubing 432 and is configured to provide fluid for irrigation to the endoscope 100. Generally, the irrigation supply tubing 432 may be a water or fluid supply line or tube for supplying water or other fluid to an endoscope.

The first and second containers 404, 422 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 404, 422 may be entirely translucent, entirely opaque, or combinations thereof. In some cases, the first and second containers 404, 422 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 404, 422 may be variable. For example, the volume of the first container 404 and/or the second container 422 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 402, 420 may be pre-filled (e.g., prior to entering the procedure suite or at the time of manufacturing) with water or other fluid. In some cases, the clinician may select the reservoir(s) 402, 420 from a plurality of differently sized available reservoirs based on the number and/or types of procedures expected for a typical or the specific day. In the illustrated embodiments, the first reservoir 402 may supply fluid to the second reservoir 420. By selecting a first reservoir 402 having a volume large enough to accommodate an entire day of procedures, the need for replacing the sterile fluid source (e.g., the first reservoir 402) may be reduced or eliminated. In some cases, the first reservoir 402 may be used to periodically refill the second reservoir 420. Thus, the volume of the first reservoir 402 may be greater than the volume of the second reservoir 420, although this is not required. It is further contemplated that, in some embodiments, one or both of the first or second reservoirs 402, 420 may be a rigid bottle.

It is contemplated that flexible bags may utilize less plastic (or other material) than a bottle designed to hold a similar amount of fluid. Thus, the use of a flexible bag as a fluid reservoir 402, 420 may increase the level of environmental sustainability of the system 400. For example, if the user sets up the system with a 3000 mL (3 liter) bag reservoir 402 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 402 may further include one or more ports 408a, 408b, such as, but not limited to a spike port or a septum port, extending from and in selective fluid communication with an interior of the first container 404. The ports 408a, 408b may be formed as a monolithic structure with the first container 404. The ports 408a, 408b may be generally tubular structures with each port 408a, 408b defining a lumen extending therethrough. The lumens of the ports 408a, 408b may be configured to selectively fluidly couple the interior of the first container 404 with another component, such as, but not limited to, a fluid or water supply tube. In some embodiments, the ports 408a, 408b may be positioned adjacent to a bottom end 412 of the first reservoir 402. However, this is not required. The ports 408a, 408b may be positioned in other locations, as desired. If the ports 408a, 408b are positioned at a location other than the bottom end 412 of the first container 404, a dip tube or tube extension may be required to access the fluid at the bottom of the first container 404. In some cases, at least one port 408b may be configured to be coupled to the first fluid inlet (or water supply tube) 426 while another port 408a may be configured to allow the user to add additives to the fluid 406. While the first reservoir 402 is illustrated as including two ports 408a, 408b, the first reservoir 402 may include one port or more than two ports, as desired.

While not explicitly shown, the ports 408a, 408b may each include a removable cap or seal configured to form a fluid tight seal with the port 408a, 408b. The removable cap or seal may help to maintain the sterility of the ports 408a, 408b. The removable cap or seal may be coupled to a free end of the ports 408a, 408b using a number of different techniques. For example, the cap or seal may be coupled to the port 408a, 408b using a threaded engagement, a friction fit, a snap fit, etc. In other instances, the cap or seal may be removed through a twisting motion configured to break the cap or seal from the port 408a, 408b. Once the cap or seal has been removed, the port 408a, 408b may be pierced with a spike tip or spike port adaptor 410 that is coupled to the first fluid inlet 426. For example, in addition to the removable cap or seal, the port 408a, 408b may include an internal seal disposed within a lumen of the port 408a, 408b that may be punctured or pierced by the spike port adaptor 410. The internal seal may be configured to prevent fluid 406 from leaking from the first container 404 prior to the spike port adaptor 410 being inserted into the port 408a, 408b. In some embodiments, the internal seal may be self-sealing such that upon removal of the spike port adaptor 410 fluid is prevented from leaking from the port 408a, 408b. The outer surface of the spike port adaptor 410 may form an interference fit with the inner surface of the port 408a, 408b. The fit and/or coupling between the spike port adaptor 410 and the port 408a, 408b may be sufficient to remain in place when the first fluid inlet 426 and the second reservoir 420 are coupled to the spike port adaptor 410. It is contemplated that the spike port adaptor 410 may be inserted into one of the ports 408a, 408b utilizing universally used aseptic techniques such as those used with IV fluid bags. This may help reduce infection risk by maintaining sterile components, not introducing contaminants into the fluid 406, etc. It is further contemplated that additives may be added to the fluid 406 using similar aseptic techniques via one of the ports 408a, 408b.

The first reservoir 402 may include a handle 416 positioned adjacent to a top portion 414 thereof. The handle 416 may define an opening or through hole 418 for receiving a hand or hook therethrough to carry the first reservoir 402. In some cases, the handle 416 may include an undulating surface configured to provide a more ergonomic grip for the user. It is contemplated that the handle 416 may be formed from a similar material as the first container 404 or a different material, as desired. In some examples, the handle 416 may be formed from polyethylene terephthalate (PET), polypropylene (PP), etc. The handle 416 may allow the first reservoir 402 to be hung from a hook, such as, but not limited to an IV stand. Hanging the first reservoir 402 may allow the first reservoir 402 to be positioned above the level of an endoscope cart which may enable the user to see the fluid 406 level at any time. This may help the clinician avoid running out of fluid during a procedure. Additionally, elevating the reservoir may eliminate the need for the clinician to bend or stoop during setup of the system 400 and/or to change the first reservoir 402. In some cases, head pressure generated from elevating the first reservoir 402 may enable rapid priming of the irrigation circuit (and/or lens wash circuit if so connected) which may save time during setup. It is further contemplated that hanging the first reservoir 402 from a hook or IV stand may allow the first reservoir 402 to be positioned away from expensive capital equipment thus reducing or eliminating the potential for fluid running or flowing inadvertently onto the capital equipment and causing damage or destruction.

The second reservoir 420 may further include one or more ports 434a, 434b, such as, but not limited to a spike port or a septum port, extending from and in selective fluid communication with an interior of the second container 422. The ports 434a, 434b may be formed as a monolithic structure with the second container 422. The ports 434a, 434b may be a generally tubular structure with the ports 434a, 434b defining a lumen extending therethrough. The lumen of the ports 434a, 434b may be configured to selectively fluidly couple the interior of the second container 422 with another component, such as, but not limited to, fluid/water/gas supply tube(s) and/or irrigation tubes. In some cases, a first port 434a, 434b may be configured to be coupled to the gas and lens wash supply tubing 428, 430 and a second port 434b may be configured to be coupled to the irrigation supply tubing 432. In some embodiments, the port 434a, 434b may be positioned adjacent to a bottom end 436 of the second reservoir 420. However, this is not required. The ports 434a, 434b may be positioned in other locations, as desired. If the ports 434a, 434b are positioned at a location other than the bottom end 436 of the second container 422, a dip tube or tube extension may be required (e.g., coupled to the lens wash supply tubing 430) to access the fluid at the bottom of the second container 422. While the second reservoir 420 is illustrated as including two ports 434a, 434b, the second reservoir 420 may include fewer than two or more than two ports, as desired.

While not explicitly shown, the ports 434a, 434b may include a removable cap or seal configured to form a fluid tight seal with the ports 434a, 434b. The removable cap or seal may help to maintain the sterility of the ports 434a, 434b. The removable cap or seal may be coupled to a free end of the ports 434a, 434b using a number of different techniques. For example, the cap or seal may be coupled to the ports 434a, 434b using a threaded engagement, a friction fit, a snap fit, etc., or may be fixedly coupled using a number of techniques such as adhesive or solvent bonding. In other instances, the cap or seal may be removed through a twisting motion configured to break the cap or seal from the ports 434a, 434b. Once the cap or seal has been removed, the ports 434a, 434b may be pierced with a spike tip or spike port adaptor (not explicitly shown) that is coupled to the gas and lens wash supply tubing 428, 430 or with a spike tip or spike port adaptor (not explicitly shown) that is coupled to the irrigation supply tubing 432. For example, in addition to the removable cap or seal, the ports 434a, 434b may include an internal seal disposed within a lumen of the ports 434a, 434b that may be punctured or pierced by the spike port adaptor. The internal seal may be configured to prevent fluid 424 from leaking from the second container 422 prior to the spike port adaptor being inserted into the ports 434a, 434b. In some embodiments, the internal seal may be self-scaling such that upon removal of the spike port adaptor fluid is prevented from leaking from the ports 434a, 434b. The outer surface of the spike port adaptor may form an interference fit with the inner surface of the ports 434a, 434b. The fit and/or coupling between the spike port adaptor and the ports 434a, 434b may be sufficient to remain in place when the gas and fluid supply tubing 428, 430, irrigation supply tubing 432, and/or other tubing sets are coupled to the spike port adaptor. It is contemplated that the spike port adaptors may be inserted into one of the ports 434a, 434b utilizing universally used aseptic techniques such as those used with IV fluid bags. This may help reduce infection risk by maintaining sterile components, not introducing contaminants into the fluid 424, etc. It is further contemplated that additives may be added to the fluid 424 using similar aseptic techniques via one of the ports 434a, 434b, if so desired. In some cases, other coupling mechanisms may be used as desired to couple the gas and lens wash supply tubing 428, 430 and/or the irrigation supply tubing 432 to the port 434a, 434b. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 428 extends from a second end external to the second container 422 to the first port 434a. While not explicitly shown, the gas supply tubing 428 may extend into the interior of the second container 422 and terminate within a reservoir gap (e.g., above the level of the fluid 424). However, in some cases, the gas supply tubing 428 may terminate within the fluid 424. A lumen extends through the gas supply tubing 428 for receiving a flow of air and/or gas therethrough. The lumen of the gas supply tubing 428 may be in operative fluid communication with a top portion of the interior of the second container 422. The lens wash supply tubing 430 extends from a second end external to the second reservoir 420 to a first end in fluid communication with a bottom portion 436 of the second container 422. In some embodiments, the lens wash supply tubing 430 may terminate at the first port 434a. A lumen extends through the lens wash supply tubing 430 for receiving a flow of fluid therethrough. The lumen of the lens wash supply 430 is in selective operative fluid communication with a bottom portion 436 of the second container 422. In the illustrated embodiment, the gas supply tubing 428 and the lens wash supply tubing 430 may couple to the second container 422 through a single or common opening (e.g., port 434a). For example, the gas supply tubing 428 and the lens wash supply tubing 430 may be coaxially arranged. However, this is not required. In some cases, the gas supply tubing 428 and the lens wash supply tubing 430 may extend in a side by side arrangement or may be separately connected to the second container 422 in different locations.

The irrigation supply tube 432 extends from a second end region external to the second container 422 and positioned within a pump head 324 of the peristaltic irrigation pump 315 to a first end coupled to the second port 434b. The first end of the irrigation supply tube 432 is coupled to the spike port adaptor which in turn is configured to extend through a lumen of the port 434b and pierce a seal within the lumen of the port 434b to fluidly couple the interior of the second container 422 with the lumen of the irrigation supply tube 432. The second end of the irrigation supply tube 432 is configured to be fluidly coupled with an irrigation lumen of the endoscope 100. When irrigation water is required, fluid is pumped from the second container 422 by operating the irrigation pump 315, such as by depressing a footswitch (not shown), and flows from the second reservoir 420, through the irrigation supply tubing 432, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in line with the downstream irrigation supply tubing 255c. The flow control valve 326 may prevent the unintentional flow of fluid from the second container 422 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the second reservoir 420 and the irrigation pump 315. The flow control valve 326 may also prevent fluid from leaking from the downstream irrigation supply tube 255c when the endoscope 100 is changed between patients and the tubing set connector is separated from the endoscope water port.

The second container 422 may further include the first fluid inlet 426 and a second fluid inlet 440. While the first and second fluid inlets 426, 440 are illustrated as being adjacent to or extending from a top portion 442 of the second container 422, the first and/or second fluid inlets 426, 440 may be positioned at other locations about the second container 422, as desired. In some embodiments, the first and/or second fluid inlets 426, 440 may be tubular members formed as a single monolithic structure with the second container 422. In other embodiments, the first and/or second fluid inlets 426, 440 may include tubular components releasably coupled to ports (similar in form and function to ports 434a, 434b) formed in or with the container 422.

The first fluid inlet 426 may be in selective fluid communication with the first reservoir 402. For example, a first end 444 of the first fluid inlet 426 may be coupled to the spike port adaptor 410 which in turn is configured to extend through a lumen of the port 408b and pierce a seal within the lumen of the port 408b to fluidly couple the interior of the first container 404 with the lumen of the first fluid inlet 426.

A flow control mechanism, such as, but not limited to, a one-way valve 446 may be positioned between the first end 444 and the second end 448 of the first fluid inlet 426 to selectively fluidly couple the second container 422 with the first container 404. The one-way valve 446 may be configured to be opened to allow fluid to selectively pass from the first reservoir 402 to the second reservoir 420 while preventing fluid (e.g., gas, water, or other fluid) from exiting the second container 422 and entering the first fluid inlet 426 and/or the first container 404. In some embodiments, the one-way valve 446 may be replaced with a clamp which may compress the first fluid inlet 426 to selectively fluidly isolate the second container 422 from the first container 404 and removed to selectively couple the second container 422 with the first container 404. In yet other embodiments, the one-way valve 446 may be replaced with a spring-loaded valve, a stopcock, or other two-way valve. When it is desired to add fluid to the second reservoir 420 from the first reservoir 402, the one-way valve 446 (or other flow control mechanism) may be opened or released. Fluid may flow through the first fluid inlet 426 into the second reservoir 420. Fluid may be added to the second container 422 while the irrigation pump 315 is running or while the irrigation pump 315 is idle, as desired.

The second fluid inlet (or gas supply tube) 440 of the second container 422 may be an alternative gas supply tubing configured to be coupled to an alternative gas supply (e.g., CO₂ hospital house gas source). The second fluid inlet 440 may extend from a second end external to the second container 422 to a first end coupled to the second container 422. The alternative gas supply may be used to pressurize the second container 422 to supply lens wash to the endoscope 100 and/or to provide insufflation. A lumen extends through the second fluid inlet 440 for receiving a flow of gas therethrough. The lumen of the second fluid inlet 440 is in operative fluid communication with a top portion of the second container 422. The flow of the CO₂ through the system 400 may be similar to that described above. For example, in the neutral state, CO₂ gas flows through the second fluid inlet 440 into the second container 422, up the gas supply tubing 428 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 440 into the second container 422, up the gas supply tubing 428 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 420 is maintained by delivering gas through the second fluid inlet 440. It is contemplated that the one-way valve 446 is in the closed configuration during delivery of the CO₂ gas to allow the container 422 to pressurize. In some instances, the one-way valve 446 may be configured to close without user intervention in response to the delivery of CO₂ to the second container 422. In some embodiments, the system 400 may include a branched connector (such as, but not limited to a “Y” or “T” connector) at the second fluid inlet 440 to allow either air or CO₂ to be used for pressurization or insufflation. Alternatively, air may be supplied to the second reservoir 420 via the gas supply tube 428 from a pressurizing pump 215 in the processing unit 210. It is further contemplated that the second fluid inlet 440 may include a pressure relief valve 450, such as, but not limited to, a 3-way stopcock, a clamp, a spring-loaded valve, or the like to vent pressure within the second container 422 and/or to block a flow of pressurized gas to the second container 422 during refilling of the second container 422, during procedure change-overs, and/or during equipment change-overs.

It is contemplated that the use of a flexible bag in place of a rigid bottle for the second reservoir 420 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 422 is fluidly isolated from the first container 404 when the one-way valve 446 is closed, it is contemplated that the clinician may replace the first reservoir 402 with a new (e.g., full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 402 with a new full bag, for example when the first reservoir 402 is empty or near empty, the user may hang the new bag near the first reservoir 402 to be replaced. The user may then disengage the spike port adaptor 410 from the port 408b and insert the spike port adaptor 410 into a port of the new bag. This may be performed without requiring the clinician to bend or stoop to access the first reservoir 402. The port 408b may self-seal to prevent fluid leaks from the first reservoir 402 as the first reservoir 402 is being replaced. This method of replacing the first reservoir 402 may have a lower risk of introducing contaminants into the systems relative to traditional bottle systems. For example, the change out method described herein may allow the first reservoir 402 to be changed out without having tubing dangling from a cap (as in a bottle system). Further, the system 400 may remain largely closed as the first reservoir 402 is changed out.

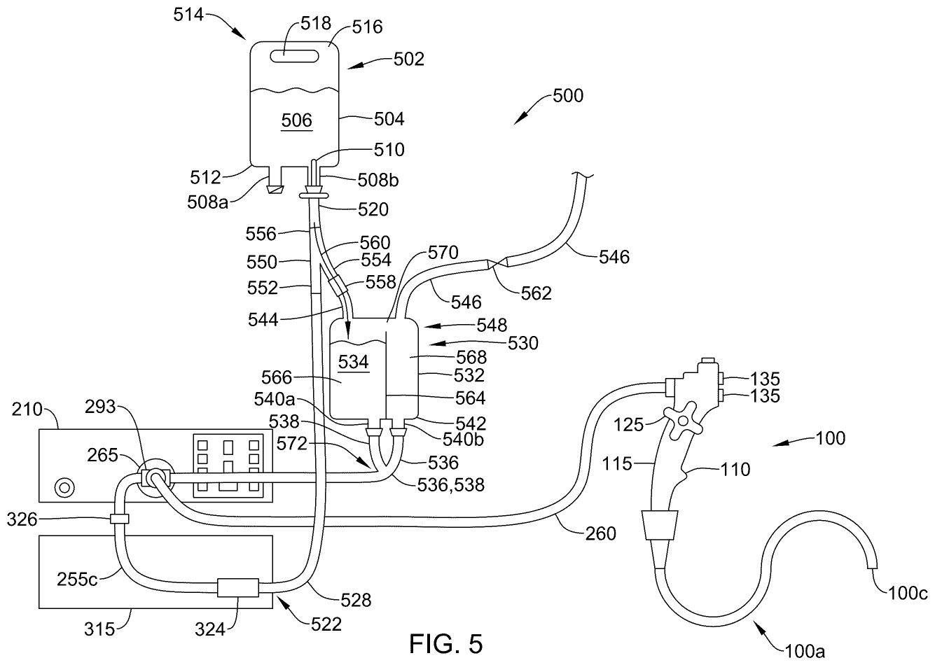

FIG. 5 depicts a schematic view of another illustrative endoscopic system 500 which may reduce the number of water reservoir changes and/or reduce opportunities for contamination during replacement of the water reservoir(s). The system 500 may include a number of advantages over the current bottle system described above. The system 500 may include components similar to the endoscope and endoscope systems described with regard to FIGS. 1-2; however, not all features may be described or shown here.

Generally, the system 500 may include a first reservoir 502 and a second reservoir 530. The first reservoir 502 may be configured to supply water or fluid for both irrigation (e.g., via the first reservoir 502) and lens wash (e.g., via the second reservoir 530). This may allow a single fluid source to be used to provide fluid for both irrigation and lens wash. While not explicitly shown, the reservoirs 502, 530 may include printed lines, numbers, or other visual indicia to allow a user to easily determine how much fluid is left in the reservoirs 502, 530.

The first reservoir 502 may include a first container 504 configured to hold a first volume of fluid 506. In the illustrated embodiment, the first container 504 is fluidly coupled to the upstream irrigation supply tubing 528 and is configured to provide fluid for irrigation to the endoscope 100. Generally, the irrigation supply tubing 528 may be a water or fluid supply line or tube for supplying water or other fluid to an endoscope. Additionally, the first container 504 may be selectively fluidly coupled to a second fluid reservoir 530. The second reservoir 530 may include a second container 532 configured to hold a second volume of fluid 534. In the illustrated embodiment, the second container 532 is fluidly coupled to the gas and lens wash supply tubing 536, 538 and is configured to provide fluid for lens wash to the endoscope 100. Generally, the lens wash supply tubing 538 may be a water or fluid supply line or tube for supplying water or other fluid to an endoscope. The gas and lens wash supply tubing 536, 538 may be coaxially arranged along at least a portion of a length thereof. For example, the gas supply tubing 536 may define a lumen that is sufficiently large in diameter to encompass a smaller diameter lens wash tubing 538, coaxially received within the gas supply tubing 536, as well as provide air to the water source in an annular space surrounding the lens wash tubing 538 to pressurize the second reservoir 530. The lens wash supply tubing 538 may be configured to exit the lumen defined by the coaxial gas supply tubing 536 in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 536, 538 may be arranged in a side-by-side arrangement.

The first and second containers 504, 532 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 504, 532 may be entirely translucent, entirely opaque, or combinations thereof. In some cases, the first and second containers 504, 532 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 504, 532 may be variable. For example, the volume of the first container 504 and/or the second container 532 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 502, 530 may be pre-filled (e.g., prior to entering the procedure suite or at the time of manufacturing) with water or other fluid. In some cases, the clinician may select the reservoir(s) 502, 530 from a plurality of differently sized available reservoirs based on the number and/or types of procedures expected for a typical or the specific day. In the illustrated embodiments, the first reservoir 502 may supply fluid to the second reservoir 530. By selecting a first reservoir 502 having a volume large enough to accommodate an entire day of procedures, the need for replacing the sterile fluid source (e.g., the first reservoir 502) may be reduced or eliminated. In some cases, the first reservoir 502 may be used to periodically refill the second reservoir 530. Thus, the volume of the first reservoir 502 may be greater than the volume of the second reservoir 530, although this is not required. It is further contemplated that, in some embodiments, one or both of the first or second reservoirs 502, 530 may be a rigid bottle.

It is contemplated that flexible bags may utilize less plastic (or other material) than a bottle designed to hold a similar amount of fluid. Thus, the use of a flexible bag as a fluid reservoir 502, 530 may increase the level of environmental sustainability of the system 500. For example, if the user sets up the system with a 3000 mL (3 liter) bag reservoir 502 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 502 may further include one or more ports 508a, 508b, such as, but not limited to a spike port or a septum port, extending from and in selective fluid communication with an interior of the first container 504. The ports 508a, 508b may be formed as a monolithic structure with the first container 504. The ports 508a, 508b may be generally tubular structures with each port 508a, 508b defining a lumen extending therethrough. The lumens of the ports 508a, 508b may be configured to selectively fluidly couple the interior of the first container 504 with another component, such as, but not limited to, a fluid or water supply tube. In some embodiments, the ports 508a, 508b may be positioned adjacent to a bottom end 512 of the first reservoir 502. However, this is not required. The ports 508a, 508b may be positioned in other locations, as desired. If the ports 508a, 508b are positioned at a location other than the bottom end 512 of the first container 504, a dip tube or tube extension may be required to access the fluid at the bottom of the first container 504. In some cases, at least one port 508b may be configured to be coupled to the upstream irrigation tubing (or water supply tube) 528 while another port 508a may be configured to allow the user to add additives to the fluid 506. While the first reservoir 502 is illustrated as including two ports 508a, 508b, the first reservoir 502 may include one port or more than two ports, as desired.

While not explicitly shown, the ports 508a, 508b may each include a removable cap or seal configured to form a fluid tight seal with the port 508a, 508b. The removable cap or seal may help to maintain the sterility of the ports 508a, 508b. The removable cap or seal may be coupled to a free end of the ports 508a, 508b using a number of different techniques. For example, the cap or seal may be coupled to the port 508a, 508b using a threaded engagement, a friction fit, a snap fit, etc. In other instances, the cap or seal may be removed through a twisting motion configured to break the cap or seal from the port 508a, 508b. Once the cap or seal has been removed, the port 508a, 508b may be pierced with a spike tip or spike port adaptor 510 that is coupled to the upstream irrigation tubing 528. For example, in addition to the removable cap or seal, the port 508a, 508b may include an internal seal disposed within a lumen of the port 508a, 508b that may be punctured or pierced by the spike port adaptor 510. The internal seal may be configured to prevent fluid 506 from leaking from the first container 504 prior to the spike port adaptor 510 being inserted into the port 508a, 508b. In some embodiments, the internal seal may be self-sealing such that upon removal of the spike port adaptor 510 fluid is prevented from leaking from the port 508a, 508b. The outer surface of the spike port adaptor 510 may form an interference fit with the inner surface of the port 508a, 508b. The fit and/or coupling between the spike port adaptor 510 and the port 508a, 508b may be sufficient to remain in place when the irrigation supply tube 528, branched connector 550, and/or other tubing sets are coupled to the spike port adaptor 510. It is contemplated that the spike port adaptor 510 may be inserted into one of the ports 508a, 508b utilizing universally used aseptic techniques such as those used with IV fluid bags. This may help reduce infection risk by maintaining sterile components, not introducing contaminants into the fluid 506, etc. It is further contemplated that additives may be added to the fluid 506 using similar aseptic techniques via one of the ports 508a, 508b.

The first reservoir 502 may include a handle 516 positioned adjacent to a top portion 514 thereof. The handle 516 may define an opening or through hole 518 for receiving a hand or hook therethrough to carry the first reservoir 502. In some cases, the handle 516 may include an undulating surface configured to provide a more ergonomic grip for the user. It is contemplated that the handle 516 may be formed from a similar material as the first container 504 or a different material, as desired. In some examples, the handle 516 may be formed from polyethylene terephthalate (PET), polypropylene (PP), etc. The handle 516 may allow the first reservoir 502 to be hung from a hook, such as, but not limited to an IV stand. Hanging the first reservoir 502 may allow the first reservoir 502 to be positioned above the level of an endoscope cart which may enable the user to see the fluid 506 level at any time. This may help the clinician avoid running out of fluid during a procedure. Additionally, elevating the reservoir may eliminate the need for the clinician to bend or stoop during setup of the system 500 and/or to change the first reservoir 502. In some cases, head pressure generated from elevating the first reservoir 502 may enable rapid priming of the irrigation circuit (and/or lens wash circuit if so connected) which may save time during setup. It is further contemplated that hanging the first reservoir 502 from a hook or IV stand may allow the first reservoir 502 to be positioned away from expensive capital equipment thus reducing or eliminating the potential for fluid running or flowing inadvertently onto the capital equipment and causing damage or destruction.

The first reservoir 502 may be connected in fluid communication with a lumen of the upstream irrigation supply tube 528. The upstream irrigation supply tube 528 extends from a second end region 522 external to the container 504 and positioned within a pump head 324 of the peristaltic irrigation pump 315 to a first end 520. The first end 520 of the upstream irrigation supply tube 528 is coupled to the spike port adaptor 510 which in turn is configured to extend through a lumen of the port 508b and pierce a seal within the lumen of the port 508b to fluidly couple the interior of the container 504 with the lumen of the upstream irrigation supply tube 528. The second end of the upstream irrigation supply tube 528 is configured to be fluidly coupled with an irrigation lumen of the endoscope 100. When irrigation water is required, fluid is pumped from the first container 504 by operating the irrigation pump 315, such as by depressing a footswitch (not shown), and flows from the first reservoir 502, through the upstream irrigation supply tubing 528 and a branched connector 550, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in line with the downstream irrigation supply tubing 255c. The flow control valve 326 may prevent the unintentional flow of fluid from the first container 504 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 502 and the irrigation pump 315. The flow control valve 326 may also prevent fluid from leaking from the downstream irrigation supply tube 255c when the endoscope 100 is changed between patients and the tubing set connector is separated from the endoscope water port.

The second reservoir 530 may further include one or more ports 540a, 540b, such as, but not limited to a spike port or a septum port, extending from and in selective fluid communication with an interior of the second container 532. The ports 540a, 540b may be formed as a monolithic structure with the second container 532. The ports 540a, 540b may be a generally tubular structure with the ports 540a, 540b defining a lumen extending therethrough. The lumen of the ports 540a, 540b may be configured to selectively fluidly couple the interior of the second container 532 with another component, such as, but not limited to, fluid/water/gas supply tube(s). In some cases, the ports 540a, 540b may be configured to be coupled to the gas and lens wash supply tubing 536, 538. For example, the first port 540a may be configured to be coupled to the lens wash supply tubing 538 and the second port 540b may be configured to be coupled to the gas supply tubing 536. In some embodiments, the ports 540a, 540b may be positioned adjacent to a bottom portion 542 of the second reservoir 530. However, this is not required. The ports 540a, 540b may be positioned in other locations, as desired. If the ports 540a, 540b is positioned at a location other than the bottom portion 542 of the second container 532, a dip tube or tube extension may be required (e.g., coupled to the lens wash supply tubing 538) to access the fluid at the bottom of the second container 532. While the second reservoir 530 is illustrated as including two ports 540a, 540b, the second reservoir 530 may include fewer than two ports or more than two ports, as desired.

While not explicitly shown, the ports 540a, 540b may include a removable cap or seal configured to form a fluid tight seal with the ports 540a, 540b. The removable cap or seal may help to maintain the sterility of the ports 540a, 540b. The removable cap or seal may be coupled to a free end of the ports 540a, 540b using a number of different techniques. For example, the cap or seal may be coupled to the ports 540a, 540b using a threaded engagement, a friction fit, a snap fit, etc., or may be fixedly coupled using a number of techniques such as adhesive or solvent bonding. In other instances, the cap or seal may be removed through a twisting motion configured to break the cap or seal from the ports 540a, 540b. Once the cap or seal has been removed, the ports 540a, 540b may be pierced with a spike tip or spike port adaptor (not explicitly shown) that is coupled to the lens wash supply tubing 538 and gas supply tubing 536, respectively. For example, in addition to the removable cap or seal, the ports 540a, 540b may include an internal seal disposed within a lumen of the ports 540a, 540b that may be punctured or pierced by the spike port adaptor. The internal seal may be configured to prevent fluid 534 from leaking from the second container 532 prior to the spike port adaptor being inserted into the ports 540a, 540b. In some embodiments, the internal seal may be self-scaling such that upon removal of the spike port adaptor fluid is prevented from leaking from the ports 540a, 540b. The outer surface of the spike port adaptor may form an interference fit with the inner surface of the ports 540a, 540b. The fit and/or coupling between the spike port adaptor and the ports 540a, 540b may be sufficient to remain in place when the gas and fluid supply tubing 536, 538 and/or other tubing sets are coupled to the spike port adaptor. It is contemplated that the spike port adaptor may be inserted into one of the ports 540a, 540b utilizing universally used aseptic techniques such as those used with IV fluid bags. This may help reduce infection risk by maintaining sterile components, not introducing contaminants into the fluid 534, etc. In some cases, other coupling mechanisms may be used as desired to couple the gas and lens wash supply tubing 536, 538 to the ports 540a, 540b. Some illustrative coupling mechanisms may include, but are not limited to, threaded engagements, snap fits, friction fits, quick connect style couplers, etc., or may be fixedly coupled using a number of techniques such as adhesive or solvent bonding.

The second container 532 may include a partition 564 disposed within an interior thereof to divide the second container 532 into a first chamber 566 configured to hold the second volume of fluid 534 and a second chamber 568 configured to receive a flow of air from the gas supply tubing 536. The first chamber 566 may have a first volume and the second chamber 568 may have a second volume. In some cases, the first volume may be greater than the second volume. Although, this is not required. In other embodiments, the first and second volumes may be approximately equal or the second volume may be greater than the first volume. In some examples, the partition 564 may be formed by heat or pressure welding the container 532 along the partition region. The partition 564 may be laterally disposed between the first port 540a and the second port 540b to fluidly isolate the gas inlet from the water (or other fluid) outlet. The partition 564 may extend from the bottom portion 542 of the second container 532 towards the first end 548 of the second container 532. The partition 564 may terminate before reaching the first end 548 of the second container 532 to define an opening 570 between the first chamber 566 and the second chamber 568. Said differently, the partition 564 may have a height less than a height of the reservoir 530. The opening 570 may allow air to travel from the gas supply tubing 536, up the second chamber 568 and to the opening 570 where the air may pressurize the entire container 532 to enable insufflation and/or lens wash. It is contemplated that the second volume of fluid 534 may be maintained at a level below the upper end of the partition 564 to preclude water or other fluid from entering the second chamber 568. When air is no longer needed, air may travel through the second chamber 568 to the gas supply tubing 536 and out the gas/water valve 140 to depressurize the second reservoir 530.

The gas supply tubing 536 extends from a second end external to the second container 532 to a first end coupled to the second port 540b. A lumen extends through the gas supply tubing 536 for receiving a flow of air and/or gas therethrough. The lumen of the gas supply tubing 536 may be in operative fluid communication with the interior of the second container 532. The lens wash supply tubing 538 extends from a second end external to the second reservoir 530 to a first end coupled to the first port 540a and in fluid communication with a bottom portion 542 of the second container 532. The gas supply tubing 536 and the lens wash supply tubing 538 be concentrically arranged up to a bifurcated port 572 exterior to and adjacent to the bottom portion 542 of the second container 532. In some examples, the bifurcated port 572 may be a “Y” connector or a “T” connector, although this is not required. At the bifurcated port 572, the lens wash supply tubing 538 may split from the gas supply tubing 536 and couple to the first port 540a and the first chamber 566. A lumen extends through the lens wash supply tubing 538 for receiving a flow of fluid therethrough. The lumen of the lens wash supply 538 is in selective operative fluid communication with a bottom portion 542 of the second container 532. It is contemplated that providing air to the second chamber 568 may eliminate the need for the gas supply tubing 536 to extend above the water line or for the lens wash supply tubing 538 to extend from the top portion 548 to the bottom portion 542 of the second container 532.

The second container 532 may further include a first fluid inlet 544 and a second fluid inlet 546. While the first and second fluid inlets 544, 546 are illustrated as being adjacent to or extending from the top portion 548 of the second container 532, the first and/or second fluid inlets 544, 546 may be positioned at other locations about the second container 532, as desired. In some embodiments, the first and/or second fluid inlets 544, 546 may be tubular members formed as a single monolithic structure with the second container 532. In other embodiments, the first and/or second fluid inlets 544, 546 may include tubular components releasably coupled to ports (similar in form and function to ports 540a, 540b) formed in or with the container 532.

The first fluid inlet 544 may be in selective fluid communication with the first reservoir 502. For example, a branched connector 550 may be positioned in-line with the upstream irrigation tubing 528. In some embodiments, the branched connector 550 may be a “Y” connector or a “T” connector having an inlet leg 556 defining a first fluid inlet, a first outlet leg 552 defining a first fluid outlet, and a second outlet leg 554 defining a second fluid outlet. However, it is contemplated that the branched connector 550 may include more than one fluid inlet and fewer than two or more than two fluid outlets, if so desired.

The branched connector 550 may be positioned in-line with the upstream irrigation tubing 528 such that the inlet leg 556 and the first outlet leg 552 are fluidly coupled with the lumen of the upstream irrigation tubing 528. Fluid may flow from the first reservoir 502, through the upstream irrigation tubing 528, through the branched connector 550 and again through the upstream irrigation tubing 528. The branched connector 550 may be positioned such that the inlet leg 556 is upstream of the outlet legs 552, 554 relative to a flow of irrigation fluid. In some embodiments, the branched connector 550 and the spike port 510 may be molded or formed as a single monolithic structure. It is contemplated that this may reduce connection points in the fluid circuit. In such an instance, the first end 520 of the irrigation supply tubing 528 may be fluidly coupled to the first outlet leg 552 of the branched connector 550.

The second outlet leg 554 may be fluidly coupled to the first fluid inlet 544 of the second reservoir 530. A flow control mechanism, such as, but not limited to, a one-way valve 558 may be positioned between the second fluid outlet of the second outlet leg 554 and the first fluid inlet 544 of the second reservoir 530 to selectively fluidly couple the second container 532 with the first container 504. The one-way valve 558 may be configured to be opened to allow fluid to selectively pass from the first reservoir 502 to the second reservoir 530 while preventing fluid (e.g., gas, water, or other fluid) from exiting the second container 532 and entering the irrigation supply tubing 528 and/or the first container 504. In some embodiments, the one-way valve 558 may be replaced with a clamp which may compress the first fluid inlet 544 to selectively fluidly isolate the second container 532 from the first container 504 and removed to selectively couple the second container 532 with the first container 504. In yet other embodiments, the one-way valve 558 may be replaced with a spring-loaded valve, a stopcock, or other two-way valve. When it is desired to add fluid to the second reservoir 530 from the first reservoir 502, the one-way valve 558 (or other flow control mechanism) may be opened or released. Fluid may then be at least partially diverted from the irrigation supply tubing 528 through the second outlet leg 554 of the branched connector 550 and into the second container 532 along flow path 560. Fluid may be added to the second container 532 while the irrigation pump 315 is running or while the irrigation pump 315 is idle, as desired.

The second fluid inlet (or gas supply tube) 546 of the second container 532 may be an alternative gas supply tubing configured to be coupled to an alternative gas supply (e.g., CO₂ hospital house gas source). The second fluid inlet 546 may extend from a second end external to the second container 532 to a first end coupled to the second container 532. The alternative gas supply may be used to pressurize the second container 532 to supply lens wash to the endoscope 100 and/or to provide insufflation. A lumen extends through the second fluid inlet 546 for receiving a flow of gas therethrough. The lumen of the second fluid inlet 546 is in operative fluid communication with a top portion of the second container 532. The flow of the CO₂ through the system 500 may be similar to that described above. For example, in the neutral state, CO₂ gas flows through the second fluid inlet 546 into the second container 532, up the gas supply tubing 536 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 546 into the second container 532, up the gas supply tubing 536 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 530 is maintained by delivering gas through the second fluid inlet 546. It is contemplated that the one-way valve 558 is in the closed configuration during delivery of the CO₂ gas to allow the container 532 to pressurize. In some instances, the one-way valve 558 may be configured to close without user intervention in response to the delivery of CO₂ to the second container 532. In some embodiments, the system 500 may include a branched connector (such as, but not limited to a “Y” or “T” connector) at the second fluid inlet 546 to allow either air or CO₂ to be used for pressurization or insufflation. Alternatively, air may be supplied to the second reservoir 530 via the gas supply tube 536 from a pressurizing pump 215 in the processing unit 210. It is further contemplated that the second fluid inlet 546 may include a pressure relief valve 562, such as, but not limited to, a 3-way stopcock, a clamp, a spring-loaded valve, or the like to vent pressure within the second container 532 and/or to block a flow of pressurized gas to the second container 532 during refilling of the second container 532, during procedure change-overs, and/or during equipment change-overs.

It is contemplated that the use of a flexible bag in place of a rigid bottle for the second reservoir 530 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 532 is fluidly isolated from the first container 504 when the one-way valve 558 is closed, it is contemplated that the clinician may replace the first reservoir 502 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 502 with a new full bag, for example when the first reservoir 502 is empty or near empty, the user may hang the new bag near the first reservoir 502 to be replaced. The user may then disengage the spike port adaptor 510 from the port 508b and insert the spike port adaptor 510 into a port of the new bag. This may be performed without requiring the clinician to bend or stoop to access the first reservoir 502. The port 508b may self-seal to prevent fluid leaks from the first reservoir 502 as the first reservoir 502 is being replaced. This method of replacing the first reservoir 502 may have a lower risk of introducing contaminants into the systems relative to traditional bottle systems. For example, the change out method described herein may allow the first reservoir 502 to be changed out without having tubing dangling from a cap (as in a bottle system). Further, the system 500 may remain largely closed as the first reservoir 502 is changed out.

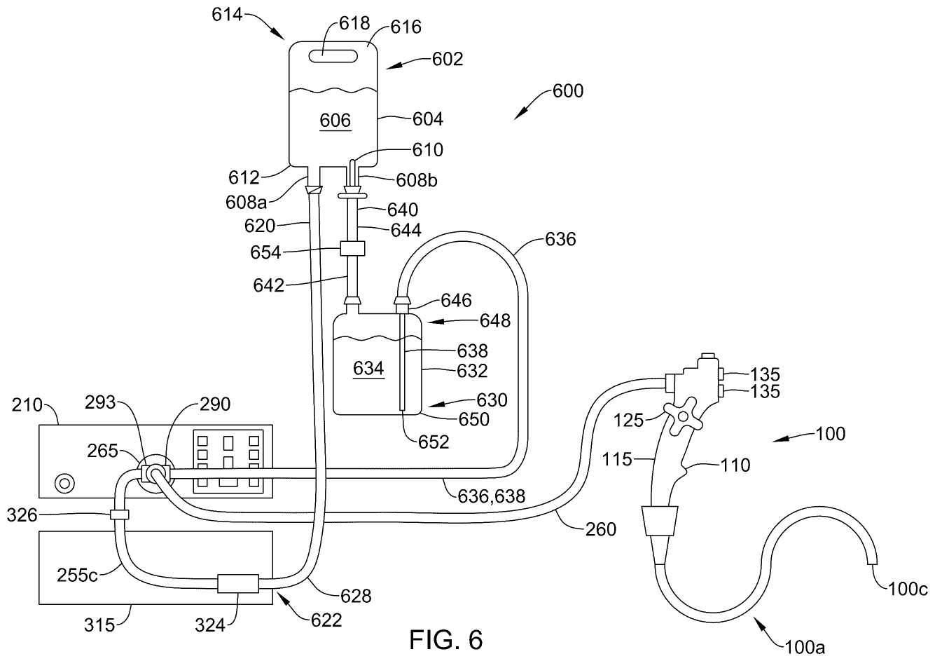

FIG. 6 depicts a schematic view of another illustrative endoscopic system 600 which may reduce the number of water reservoir changes and/or reduce opportunities for contamination during replacement of the water reservoir(s). The system 600 may include a number of advantages over the current bottle system described above. The system 600 may include components similar to the endoscope and endoscope systems described with regard to FIGS. 1-2; however, not all features may be described or shown here.

Generally, the system 600 may include a first reservoir 602 and a second reservoir 630. The first reservoir 602 may be configured to supply water or fluid for both irrigation (e.g., via the first reservoir 602) and lens wash (e.g., via the second reservoir 630). This may allow a single fluid source to be used to provide fluid for both irrigation and lens wash. While not explicitly shown, the reservoirs 602, 630 may include printed lines, numbers, or other visual indicia to allow a user to easily determine how much fluid is left in the reservoirs 602, 630.

The first reservoir 602 may include a first container 604 configured to hold a first volume of fluid 606. In the illustrated embodiment, the first container 604 may be selectively fluidly coupled to a first fluid inlet 640 of the second reservoir 630. Generally, the first fluid inlet 640 may be a water or fluid supply line or tube for supplying water or other fluid to the second reservoir 630. Additionally, the first container 604 is fluidly coupled to the irrigation supply tubing 628 and is configured to provide fluid for irrigation to the endoscope 100. Generally, the irrigation supply tubing 628 may be a water or fluid supply line or tube for supplying water or other fluid to an endoscope.

The second reservoir 630 may include a second container 632 configured to hold a second volume of fluid 634. In the illustrated embodiment, the second container 632 is fluidly coupled to the gas and lens wash supply tubing 636, 638 and is configured to provide fluid for lens wash to the endoscope 100. Generally, the lens wash supply tubing 638 may be a water or fluid supply line or tube for supplying water or other fluid to an endoscope. The gas and lens wash supply tubing 636, 638 may be coaxially arranged. For example, the gas supply tubing 636 may define a lumen that is sufficiently large in diameter to encompass a smaller diameter lens wash tubing 638, coaxially received within the gas supply tubing 636, as well as provide air to the water source in an annular space surrounding the lens wash tubing to pressurize the second reservoir 630. The lens wash supply tubing 638 may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 636, 638 may be arranged in a side-by-side arrangement.

The first and second containers 604, 632 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 604, 632 may be entirely translucent, entirely opaque, or combinations thereof. In some cases, the first and second containers 604, 632 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 604, 632 may be variable. For example, the volume of the first container 604 and/or the second container 632 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 602, 630 may be pre-filled (e.g., prior to entering the procedure suite or at the time of manufacturing) with water or other fluid. In some cases, the clinician may select the reservoir(s) 602, 630 from a plurality of differently sized available reservoirs based on the number and/or types of procedures expected for a typical or the specific day. In the illustrated embodiments, the first reservoir 602 may supply fluid to the second reservoir 630. By selecting a first reservoir 602 having a volume large enough to accommodate an entire day of procedures, the need for replacing the sterile fluid source (e.g., the first reservoir 602) may be reduced or eliminated. In some cases, the first reservoir 602 may be used to periodically refill the second reservoir 630. Thus, the volume of the first reservoir 602 may be greater than the volume of the second reservoir 630, although this is not required. It is further contemplated that, in some embodiments, one or both of the first or second reservoirs 602, 630 may be a rigid bottle.

The first reservoir 602 may further include one or more ports 608a, 608b, such as, but not limited to a spike port or a septum port, extending from and in selective fluid communication with an interior of the first container 604. In some cases, the first port 608a may be a septum port and the second port 608b may be a spike port. However, this is not required. The configuration may be reversed or both ports 608a, 608b may be of the same type. The ports 608a, 608b may be formed as a monolithic structure with the first container 604. The ports 608a, 608b may be generally tubular structures with each port 608a, 608b defining a lumen extending therethrough. The lumens of the ports 608a, 608b may be configured to selectively fluidly couple the interior of the first container 604 with another component, such as, but not limited to, a fluid or water supply tube. In some embodiments, the ports 608a, 608b may be positioned adjacent to a bottom end 612 of the first reservoir 602. However, this is not required. The ports 608a, 608b may be positioned in other locations, as desired. If the ports 608a, 608b are positioned at a location other than the bottom end 612 of the first container 604, a dip tube or tube extension may be required to access the fluid at the bottom of the first container 604. In some cases, at least one port 608b may be configured to be coupled to the first fluid inlet (or water supply tube) 640 while another port 608a may be configured to allow the user to add additives to the fluid 606. While the first reservoir 602 is illustrated as including two ports 608a, 608b, the first reservoir 602 may include one port or more than two ports, as desired.

While not explicitly shown, the ports 608a, 608b may each include a removable cap or seal configured to form a fluid tight seal with the port 608a, 608b. The removable cap or seal may help to maintain the sterility of the ports 608a, 608b. The removable cap or seal may be coupled to a free end of the ports 608a, 608b using a number of different techniques. For example, the cap or seal may be coupled to the port 608a, 608b using a threaded engagement, a friction fit, a snap fit, etc. In other instances, the cap or seal may be removed through a twisting motion configured to break the cap or seal from the port 608a, 608b. Once the cap or seal has been removed, the port 608a, 608b may be pierced with a spike tip or spike port adaptor(s) 610, or other connection member, such as a needle, or the like, that is coupled to the upstream irrigation supply tube 628 or the fluid supply line 640. In addition to the removable cap or seal, the port 608a, 608b may include an internal seal disposed within a lumen of the port 608a, 608b that may be punctured or pierced by the spike port adaptor(s) 610. The internal seal may be configured to prevent fluid 606 from leaking from the first container 604 prior to the spike port adaptor(s) 610, or other connection member, being inserted into the port 608a, 608b. In some embodiments, the internal seal may be self-sealing such that upon removal of the spike port adaptor(s) 610, or other connection member, fluid is prevented from leaking from the port 608a, 608b. The outer surface of the spike port adaptor(s) 610 may form an interference fit with the inner surface of the port 608a, 608b. The fit and/or coupling between the spike port adaptor(s) 610 and the port 608a, 608b may be sufficient to remain in place when the irrigation supply tube 628, the fluid supply tube 640, and/or other tubing sets are coupled to the spike port adaptor 610 or other connection member. It is contemplated that the spike port adaptor(s) 610 or other connection member may be inserted into the ports 608a, 608b utilizing universally used aseptic techniques such as those used with IV fluid bags. This may help reduce infection risk by maintaining sterile components, not introducing contaminants into the fluid 606, etc. It is further contemplated that additives may be added to the fluid 606 using similar aseptic techniques via one of the ports 608a, 608b.

The first reservoir 602 may include a handle 616 positioned adjacent to a top portion 614 thereof. The handle 616 may define an opening or through hole 618 for receiving a hand or hook therethrough to carry the first reservoir 602. In some cases, the handle 616 may include an undulating surface configured to provide a more ergonomic grip for the user. It is contemplated that the handle 616 may be formed from a similar material as the first container 604 or a different material, as desired. In some examples, the handle 616 may be formed from polyethylene terephthalate (PET), polypropylene (PP), etc. The handle 616 may allow the first reservoir 602 to be hung from a hook, such as, but not limited to an IV stand. Hanging the first reservoir 602 may allow the first reservoir 602 to be positioned above the level of an endoscope cart which may enable the user to see the fluid 606 level at any time. This may help the clinician avoid running out of fluid during a procedure. Additionally, elevating the reservoir may eliminate the need for the clinician to bend or stoop during setup of the system 600 and/or to change the first reservoir 602. In some cases, head pressure generated from elevating the first reservoir 602 may enable rapid priming of the irrigation circuit (and/or lens wash circuit if so connected) which may save time during setup. It is further contemplated that hanging the first reservoir 602 from a hook or IV stand may allow the first reservoir 602 to be positioned away from expensive capital equipment thus reducing or eliminating the potential for fluid running or flowing inadvertently onto the capital equipment and causing damage or destruction.

The second reservoir 630 may further include one or more ports 646, such as, but not limited to a spike port or a septum port, extending from and in selective fluid communication with an interior of the second container 632. The port 646 may be formed as a monolithic structure with the second container 632. The port 646 may be a generally tubular structure with the port 646 defining a lumen extending therethrough. The lumen of the port 646 may be configured to selectively fluidly couple the interior of the second container 632 with another component, such as, but not limited to, fluid/water/gas supply tube(s) and/or irrigation tubes. In some cases, the port 646 may be configured to be coupled to the gas and lens wash supply tubing 636, 638. In some embodiments, the port 646 may be positioned adjacent to a top end 648 of the second reservoir 630. However, this is not required. The ports 646 may be positioned in other locations, as desired. If the ports 646 are positioned at a location other than the bottom end 650 of the second container 632, a dip tube or tube extension may be required (e.g., coupled to the lens wash supply tubing 638) to access the fluid at the bottom of the second container 632, as can be seen in FIG. 6. While the second reservoir 630 is illustrated as including one port 646, the second reservoir 630 may include more than one port, as desired.

While not explicitly shown, the port 646 may include a removable cap or seal configured to form a fluid tight seal with the port 646. The removable cap or seal may help to maintain the sterility of the port 646. The removable cap or seal may be coupled to a free end of the port 646 using a number of different techniques. For example, the cap or seal may be coupled to the port 646 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 646. Once the cap or seal has been removed, the port 646 may be pierced with a spike tip or spike port adaptor (not explicitly shown) that is coupled to the gas and lens wash supply tubing 636, 638. For example, in addition to the removable cap or seal, the port 646 may include an internal seal disposed within a lumen of the port 646 that may be punctured or pierced by the spike port adaptor. The internal seal may be configured to prevent fluid 634 from leaking from the second container 632 prior to the spike port adaptor being inserted into the port 646. In some embodiments, the internal seal may be self-scaling such that upon removal of the spike port adaptor fluid is prevented from leaking from the port 646. The outer surface of the spike port adaptor may form an interference fit with the inner surface of the port 646. The fit and/or coupling between the spike port adaptor and the port 646 may be sufficient to remain in place when the gas and fluid supply tubing 636, 638 and/or other tubing sets are coupled to the spike port adaptor. It is contemplated that the spike port adaptors may be inserted the port 646 utilizing universally used aseptic techniques such as those used with IV fluid bags. This may help reduce infection risk by maintaining sterile components, not introducing contaminants into the fluid 634, etc. It is further contemplated that additives may be added to the fluid 634 using similar aseptic techniques via the port 646, if so desired. In some cases, other coupling mechanisms may be used as desired to couple the gas and lens wash supply tubing 636, 638 to the port 646. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 636 extends from a second end external to the second container 632 to the port 646. While not explicitly shown, the gas supply tubing 636 may extend into the interior of the second container 632 and terminate within a reservoir gap (e.g., above the level of the fluid 634). However, in some cases, the gas supply tubing 636 may terminate within the fluid 634. A lumen extends through the gas supply tubing 636 for receiving a flow of air and/or gas therethrough. The lumen of the gas supply tubing 636 may be in operative fluid communication with a top portion of the interior of the second container 632. The lens wash supply tubing 638 extends from a second end external to the second reservoir 630 to a first end 652 in fluid communication with a bottom portion 650 of the second container 632. In some embodiments, the lens wash supply tubing 638 may terminate at the first port 646. A lumen extends through the lens wash supply tubing 638 for receiving a flow of fluid therethrough. The lumen of the lens wash supply 638 is in selective operative fluid communication with a bottom portion 650 of the second container 632. In the illustrated embodiment, the gas supply tubing 636 and the lens wash supply tubing 638 may couple to the second container 632 through a single or common opening (e.g., port 646). For example, the gas supply tubing 636 and the lens wash supply tubing 638 may be coaxially arranged. However, this is not required. In some cases, the gas supply tubing 636 and the lens wash supply tubing 638 may extend in a side by side arrangement or may be separately connected to the second container 632 in different locations.

The first reservoir 602 may be connected in fluid communication with a lumen of an upstream irrigation supply tube 628. The upstream irrigation supply tube 628 extends from a second end region 622 external to the first container 604 and positioned within a pump head 324 of the peristaltic irrigation pump 315 to a first end 620. The first end 620 is coupled to a spike port adaptor, other adaptor, or connection member (not explicitly shown) which in turn is configured to extend through a lumen of the port 608a and pierce a seal within the lumen of the port 608a to fluidly couple the interior of the first container 604 with the lumen of the upstream irrigation supply tube 628. The second end of the upstream irrigation supply tube 628 is configured to be fluidly coupled with an irrigation lumen of the endoscope 100. When irrigation water is required, fluid is pumped from the first container 604 by operating the irrigation pump 315, such as by depressing a footswitch (not shown), and flows from the first reservoir 602, through the upstream irrigation supply tubing 628, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in-line with the downstream irrigation supply tubing 255c. The flow control valve 326 may prevent the unintentional flow of fluid from the first container 604 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 602 and the irrigation pump 315. The flow control valve 326 may also prevent fluid from leaking from the downstream irrigation supply tube 255c when the endoscope 100 is changed between patients.

The second container 632 may further include the first fluid inlet 640. While the first fluid inlet 640 is illustrated as being adjacent to or extending from a top portion 648 of the second container 632, the first fluid inlet 640 may be positioned at other locations about the second container 632, as desired. In some embodiments, the first fluid inlet 640 may be a tubular member formed as a single monolithic structure with the second container 632. In other embodiments, the first fluid inlet 640 may include tubular components releasably coupled to ports (similar in form and function to port 646) formed in or with the container 632.

The first fluid inlet 640 may be in selective fluid communication with the first reservoir 602. For example, a first end 644 of the first fluid inlet 640 may be coupled to the spike port adaptor 610 which in turn is configured to extend through a lumen of the port 608b and pierce a seal within the lumen of the port 608b to fluidly couple the interior of the first container 604 with the lumen of the first fluid inlet 640.

A flow control mechanism, such as, but not limited to, a one-way valve 654 may be positioned between the first end 644 and the second end 642 of the first fluid inlet 640 to selectively fluidly couple the second container 632 with the first container 604. The one-way valve 654 may be configured to be opened to allow fluid to selectively pass from the first reservoir 602 to the second reservoir 630 while preventing fluid (e.g., gas, water, or other fluid) from exiting the second container 632 and entering the first fluid inlet 640 and/or the first container 604. In some embodiments, the one-way valve 654 may be replaced with a clamp which may compress the first fluid inlet 640 to selectively fluidly isolate the second container 632 from the first container 604 and removed to selectively couple the second container 632 with the first container 604. In yet other embodiments, the one-way valve 654 may be replaced with a spring-loaded valve, a stopcock, or other two-way valve. When it is desired to add fluid to the second reservoir 630 from the first reservoir 602, the one-way valve 654 (or other flow control mechanism) may be opened or released. Fluid may flow through the first fluid inlet 640 into the second reservoir 630. Fluid may be added to the second container 632 while the irrigation pump 315 is running or while the irrigation pump 315 is idle, as desired.

While not explicitly shown, the second reservoir 630 may further include a second fluid inlet (or gas supply tube) configured to be coupled to an alternative gas supply (e.g., CO₂ hospital house gas source). When so provided, the second fluid inlet may extend from a second end external to the second container 632 to a first end coupled to the second container 632. The alternative gas supply may be used to pressurize the second container 632 to supply lens wash to the endoscope 100 and/or to provide insufflation. A lumen extends through the second fluid inlet for receiving a flow of gas therethrough. The lumen of the second fluid inlet is in operative fluid communication with a top portion of the second container 632. The flow of the CO₂ through the system 600 may be similar to that described above. It is further contemplated that the second fluid inlet may include a pressure relief valve, such as, but not limited to, a 3-way stopcock, a clamp, or a spring-loaded valve, to vent pressure within the second container 632 and/or to block a flow of pressurized gas to the second container 632 during refilling of the second container 632, during procedure change-overs, and/or during equipment change-overs.

It is contemplated that the use of a flexible bag in place of a rigid bottle for the second reservoir 630 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 632 is fluidly isolated from the first container 604 when the one-way valve 654 is closed, it is contemplated that the clinician may replace the first reservoir 602 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 602 with a new full bag, for example when the first reservoir 602 is empty or near empty, the user may hang the new bag near the first reservoir 602 to be replaced. The user may then disengage the spike port adaptor 610 from the port 608b and insert the spike port adaptor 610 into a port of the new bag. The user may also disengage the spike port adaptor (or other adaptor) from the first port 608a and insert the spike port adaptor into a port of the new bag. This may be performed without requiring the clinician to bend or stoop to access the first reservoir 602. The port 608b may self-seal to prevent fluid leaks from the first reservoir 602 as the first reservoir 602 is being replaced. This method of replacing the first reservoir 602 may have a lower risk of introducing contaminants into the systems relative to traditional bottle systems. For example, the change out method described herein may allow the first reservoir 602 to be changed out without having tubing dangling from a cap (as in a bottle system). Further, the system 600 may remain largely closed as the first reservoir 602 is changed out.

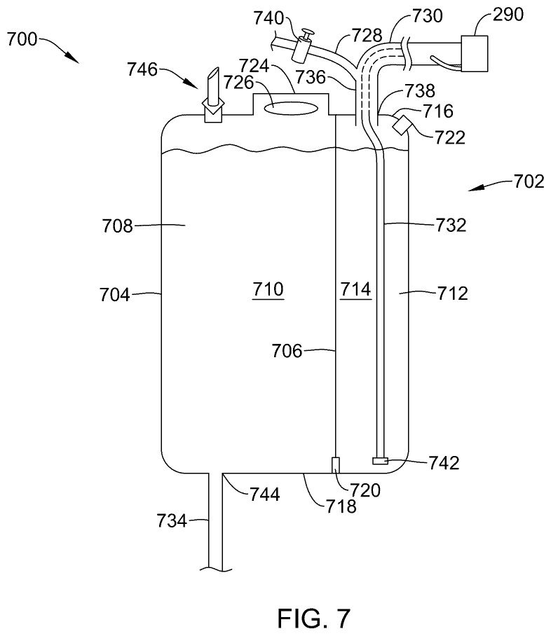

FIG. 7 depicts a schematic view of another illustrative container and tube set 700 arranged and configured to couple to an endoscope for use in an endoscopic procedure which may reduce the number of water reservoir changes and/or reduce opportunities for contamination during replacement of the water reservoir(s). The container and tube set 700 may include a number of advantages over the current bottle system described above. The container and tube set 700 may include components similar to the endoscope and endoscope systems described with regard to FIGS. 1-2; however, not all features may be described or shown here.

Generally, the container and tube set 700 may include a first reservoir 702. The first reservoir 702 may be configured to supply water or fluid for both irrigation and lens wash. This may allow a single fluid source to be used to provide fluid for both irrigation and lens wash. While not explicitly shown, the reservoir 702 may include printed lines, numbers, or other visual indicia to allow a user to easily determine how much fluid is left in the reservoir 702.

The reservoir 702 includes a receptacle or container 704 defining a first receptacle or chamber 708 for holding a first volume of fluid 710 and a second receptacle or chamber 712 for holding a second volume of fluid 714. The first and second chambers 708, 712 may be fluidly isolated from one another via a partition or seal 706 which extends from and contacts a top portion 716 to and contacting the bottom portion 718 of the container 704. The seal 706 may be permanent (e.g., not intended to open and close) or reversible (e.g., intended to open or close). For example, a permanent seal 706 may be provided by heat or pressure welding the container 704 along the seal 706. This is just one example. Other methods of creating a seal may be used as desired. In another example, waterproof hook and loop closures or zip-style closures may be used to provide a reversible seal 706. A one-way valve 720 may be molded or otherwise provided within the seal 706. The one-way valve 720 may be configured to allow a flow of fluid from the first chamber 708 to the second chamber 712 while precluding a flow of fluid from the second chamber 712 to the first chamber 708.

The first container 704 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first container 704 may be entirely translucent, entirely opaque, or combinations thereof. In some cases, the first container 704 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first container 704 may be variable. For example, the volume of the first container 704 may be 500 milliliters (mL) or greater, 1000 mL or greater, 2000 mL or greater, 3000 mL, 4000 mL or greater, etc. The volume may be less than 500 mL or greater than 4000 mL, as desired. The first reservoir 702 may be pre-filled (e.g., prior to entering the procedure suite or at the time of manufacturing) with water or other fluid. In some cases, the clinician may select the reservoir 702 from a plurality of differently sized available reservoirs based on the number and/or types of procedures expected for a typical or the specific day. By selecting a first reservoir 702 having a volume large enough to accommodate an entire day of procedures, the need for replacing or refilling the sterile fluid source (e.g., the first reservoir 702) may be reduced or eliminated. It is further contemplated that, in some embodiments, the first reservoir 702 may be a rigid bottle.

The first reservoir 702 may include a port 746 positioned adjacent the top portion 716 of the container 704. Portions of the port 746 may extend into the first chamber 708. In some embodiments, the port 746 may include a spike port adaptor configured to be received within the port of a second reservoir. For example, when the first reservoir 702 is empty or in need of additional water or fluid, the spike port adaptor may be inserted into the port of a fluid source. Illustrative fluid sources may include reservoirs similar in form and function to the reservoirs 302, 330, 402, 502, 602 described herein. Alternatively, the fluid source may be a rigid bottle. Water or fluid may flow from the fluid source, through the port 746 and into the first chamber 708. The water or fluid may flow from the first chamber 708 into the second chamber 712. In some examples, the port 746 may be provided with a removable cap to place the fluid source in selective fluid communication with the first chamber 708. In other examples, the port 746 may remain open to allow the container and tube set 700 access to atmospheric pressure. It is contemplated that the port 746 may include a filter to prevent contaminants from the atmosphere from entering the container 704.

Generally, the first and second chambers 708, 712 may provide separate fluid sources for lens wash and irrigation. For example, the first chamber 708 may provide fluid for irrigation while the second chamber 712 may provide fluid for lens wash. Having two separate chambers or chambers 708, 712 may improve the time required to pressurize the container 704 to enable lens washing capability. For example, if fluid for both lens wash and irrigation are drawn from the same source or container, the entire container must be pressurized to utilize the lens wash which may cause a lag between the input to lens wash and the function of actually washing the lens. It is contemplated that the first chamber 708 may be utilized for irrigation while the second chamber 712 may use utilized for insufflation and lens wash. The reverse configuration is also contemplated. The second chamber 712 may have a volume that is less than a volume of the first chamber 708. However, this is not required. In other instances, the volumes of the first and second chambers 708, 712 may be approximately the same or the second chamber 712 may be larger than the first chamber 708. In some embodiments, a pressure relief valve 722 may be molded into or otherwise provided in the second chamber 712, although this is not required.

The reservoir 702 may include a carrying handle or other carrying mechanism 724. The handle 724 may define an opening or through hole 726 for receiving a hand or hook therethrough to carry the reservoir 702. In some cases, the carrying handle 724 may include an ergonomic grip for the user. It is contemplated that the handle 724 may be formed from a different material from a same material as the container 704. In some examples, the handle may be formed from polyethylene terephthalate (PET), polypropylene (PP), etc.

The reservoir 702 may be connected in fluid communication with a gas supply tubing 730 and/or an alternate gas supply tubing 728, a lens wash supply tubing 732, and an irrigation supply tubing 734. The gas supply tubing 730 and the alternative gas supply tubing 728 may join prior to fluidly coupling with the container 704 to form a shared gas supply tubing 736. In some cases, the alternative gas supply tubing 728 maybe joined with the gas supply tubing 730 using a “Y” or “T” connector. The gas supply tubing 730 extends from a second end external to the reservoir 702 to the shared gas supply tubing 736 and through or to a reservoir opening 738 adjacent the top portion 716 of the second chamber 712. The shared gas supply tubing 736 may terminate within a reservoir gap, at or below the opening 738, but not extending into the remaining fluid 714 in the second chamber 712. However, in some cases, the shared gas supply tubing 736 may extend into the fluid. For example, the opening 738 may be at a bottom or side of the container 704 such that the shared gas supply tubing 736 terminates within the fluid with gas bubbling up through the fluid 714 to pressurize the second chamber 712. A lumen extends through the shared gas supply tubing 736 for receiving a flow of air and/or gas therethrough. The lumen of the shared gas supply tubing 736 is in operative fluid communication a lumen of the gas supply tubing 730, a lumen of the alternative gas supply tubing 728, and with a top portion 716 of the second chamber 712. The opening 738 in the container 704 may be a port configured to receive a spike port adaptor or may be configured to otherwise couple (e.g., a threaded engagement, a friction fit, a snap fit, etc.) with the shared gas supply tubing 736. It is further contemplated that the gas supply tubing 730 and the alternative gas supply tubing 728 may fluidly couple to the container 704 at separate openings. The alternative gas supply tubing 728 may include a pressure relief valve 740, such as, but not limited to, a 3-way stopcock, a clamp, a spring-loaded valve, or the like to vent pressure within the second chamber 712 and/or to block a flow of pressurized gas to the second chamber 712 during refilling of the second chamber 712, during procedure change-overs, and/or during equipment change-overs.

The lens wash supply tubing 732 extends from a second end external to the reservoir 702 through the reservoir opening 738, to a first end within the remaining fluid 714 at or substantially at a bottom portion 718 of the container 704. In some instances, the first end of the lens wash supply tubing 732 may include a weight 742 configured to hold the first end of the lens wash supply tubing 732 near the bottom portion 718 of the container 704. In some embodiments, the lens wash supply tubing 732 may terminate at the opening 738. For example, when the opening 738 is at or adjacent to the bottom portion 718 of the container 704 a dip tube may not be required. A lumen extends through the lens wash supply tubing 732 for receiving a flow of fluid therethrough. The lumen of the lens wash supply 732 is in selective operative fluid communication with the bottom portion of the second chamber 712. In the illustrated embodiment, the gas supply tubing 730 and the lens wash supply tubing 732 may enter the container 704 through a single or common opening 738. For example, the gas supply tubing 730 and the lens wash supply tubing 732 may be coaxially arranged. The lens wash supply tubing 732 may be configured to exit the lumen defined by the coaxial gas supply tubing 730 in any suitable sealed manner, such as, for example, an aperture, fitting, collar, and/or the like, for the purpose of transitioning from the coaxial arrangement to a side-by-side arrangement at the detachable gas/lens wash connection 290 to the endoscope connector portion 265. However, this is not required. In some cases, the gas supply tubing 730 and the lens wash supply tubing 732 may extend in a side by side arrangement or may be separately connected to the container 704 in different locations. In some embodiments, the opening 738 may include a grommet or heat seal configured to seal the container 704 about the tubing 730, 732 in a fluid and pressure tight manner.

In some embodiments, the reservoir 702 may be connected in fluid communication with a lumen of an irrigation supply tube 734. The irrigation supply tubing 734 extends from a second end to a first end which extends through an opening 744 adjacent to the bottom portion 718 of the container 704 and in fluid communication with the first chamber 708. In the use configuration, the second end of the irrigation supply tubing 734 may be external to the reservoir 702. For example, the second end of the irrigation supply tubing 734 may be disposed within the pump head of a peristaltic pump. A lumen extends through the irrigation supply tube 734 for receiving a flow of fluid therethrough. The first end of the irrigation supply tube 734 is in selective fluid communication with the bottom portion 718 of the first chamber 708. The opening 744 may include a grommet or heat seal configured to seal the container 704 about the tubing 734 in a fluid and pressure tight manner. In other embodiments, the opening 744 in the container 704 may be a port configured to receive a spike port adaptor or may be configured to otherwise couple (e.g., a threaded engagement, a friction fit, a snap fit, etc.) with the irrigation supply tubing 734. In yet other embodiments, the irrigation supply tubing 734 may be formed as monolithic structure with the container 704.

The second ends of the gas supply tubing 730 and the lens wash supply tubing 732 may be connected in fluid communication with the endoscope at gas/lens wash connection on the connector portion 265 of the umbilical 260. The gas supply tubing 730 is connected in fluid communication with a gas pump (not explicitly shown) and gas feed line (not explicitly shown), and the lens wash supply tubing 732 is connected in fluid communication with lens wash feed line (not explicitly shown), within connector portion 265. The irrigation tubing 734 is connected in fluid communication with the irrigation supply line (not explicitly shown) via an irrigation pump.

It is contemplated that the reservoir 702 may be filled and refilled as needed by fluidly coupling the port 746 with a fluid source, as described above. Further, fluid may transfer from the first chamber 708 to the second chamber 712 when the second chamber 712 is depressurized. For example, when the second chamber 712 is depressurized (e.g., by actuating one of the pressure relief valves 722, 740), fluid from the first chamber 708 may flow through the one-way valve 720 and into the second chamber 712. Flow may automatically stop when the first and second chambers 708, 712 reach an equilibrium. This may be done without requiring a user to actuate the one-way valve 720. The refilling of the reservoir 702 may be performed during a procedure or between procedures, as necessary. Refilling the reservoir 702 via the port 746 may also remove the need to disconnect the reservoir 702 from the tubing 730, 732, 734 throughout the day eliminating or greatly reducing the possibility of cross contamination by removing the need to replace the water reservoir 702.

Further, while not explicitly shown the container and tube set 700 may be used with other endoscopic systems described herein. For example, the container and tube set 700 may be used as the second reservoir 420, 530 in the systems 400, 500 of FIGS. 4 and 5. This is just one example.

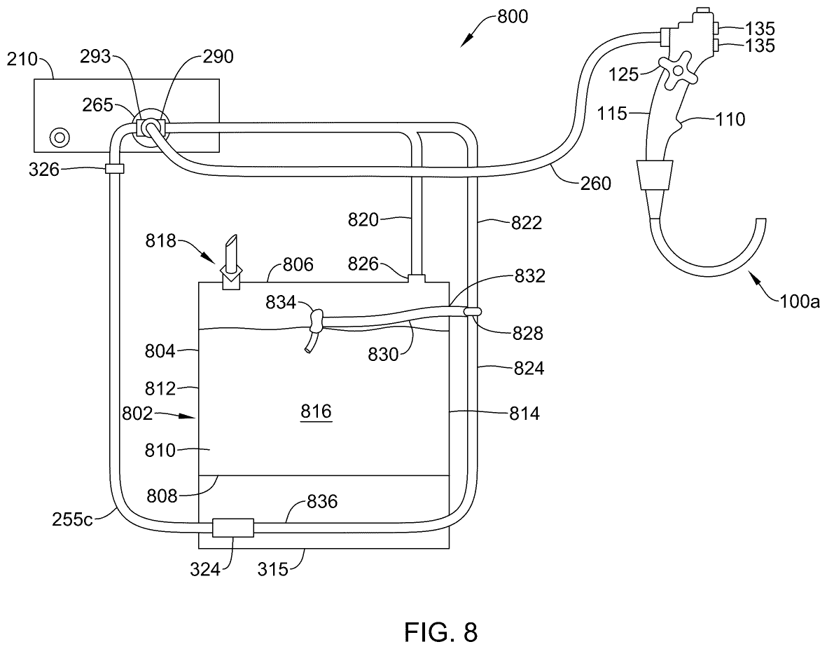

FIG. 8 depicts a schematic view of another illustrative endoscopic system 800 which may reduce the number of water reservoir changes and/or reduce opportunities for contamination during replacement of the water reservoir(s). Further, the system 800 may leverage existing pathways and resources available in the endoscopy suit. In some cases, the system 800 may reduce a length of the irrigation supply tubing 255c, the gas supply tubing 240c, and/or the lens wash supply tubing 245c. The system 800 may include components similar to the endoscope and endoscope systems described with regard to FIGS. 1-2; however, not all features may be described or shown here.

Generally, the system 800 may include a first reservoir 802. The first reservoir 802 may be configured to hold a first volume of water or fluid 816 to supply water or fluid for both irrigation and lens wash. This may allow a single fluid source to be used to provide fluid for both irrigation and lens wash. While not explicitly shown, the reservoir 802 may include printed lines, numbers, or other visual indicia to allow a user to easily determine how much fluid is left in the reservoir 802. The volume of the first container 804 may be variable. For example, the volume of the first container 804 may be 500 milliliters (mL) or greater, 1000 mL or greater, 2000 mL or greater, 3000 mL, 4000 mL or greater, etc. The volume may be less than 500 mL or greater than 4000 mL, as desired. The first reservoir 802 may be pre-filled (e.g., prior to entering the procedure suite or at the time of manufacturing) with water or other fluid.

The reservoir 802 may be formed from a rigid material that holds its shape in the absence of a fluid within the interior thereof. The container 804 extends from a top end 806 to an opposing bottom end 808. The illustrative container 804 includes a front side 810, a back side (not explicitly shown), and at least a first side 812, and a second opposing side 814. The first and second sides 812, 814 may each extend from or between the front 810 to the back. The top and bottom 806, 808 may extend from or between the first and second sides 812, 814. The reservoir 802 may be sized and/or shaped for the bottom surface 808 thereof to rest on the irrigation pump 315. However, this is not required. The use of the terms “front”, “back”, “first”, “second”, “top”, and “bottom” are not intended to limit the container 804 to a particular orientation, but rather facilitate discussion of relative orientation. Further, the container 804 is not limited to a rectangular prism or a generally rectangular structure. Other shapes may be used for the container 804, as desired.

The first reservoir 802 may include a port 818 positioned adjacent the top 806 of the container 804. Portions of the port 818 may extend into the interior of the container 804. In some embodiments, the port 818 may include a spike port adaptor configured to be received within the port of a second reservoir. For example, when the first reservoir 802 is empty or in need of additional water or fluid, the spike port adaptor may be inserted into the port of a fluid source. Illustrative fluid sources may include reservoirs similar in form and function to the reservoirs 302, 330, 402, 502, 602 described herein. Alternatively, the fluid source may be a rigid bottle. Water or fluid may flow from the fluid source, through the port 818 and into the interior of the container 804. In some examples, the port 818 may be provided with a removable cap to place the fluid source in selective fluid communication with the container 804. The cap may remain in place during use of the system 800 to allow the container 804 to be pressurized to deliver insufflation or lens wash fluid. In some embodiments, the cap may be removed to allow the system 800 to depressurize or to access to atmospheric pressure.

The reservoir 802 may be connected in fluid communication with a gas supply tubing 820 and/or an alternate gas supply tubing (not explicitly shown), a lens wash supply tubing 822, and an upstream irrigation supply tubing 824. When so provided, the alternative gas supply tubing may join the gas supply tubing 820 prior to fluidly coupling with the container 804 to form a shared gas supply tubing or may enter the container 804 through a separate opening. The gas supply tubing 820 extends from a second end external to the reservoir 802 and through or to a reservoir opening 826 adjacent the top 806 of the container 804. The gas supply tubing 820 may terminate within a reservoir gap, at or below the opening 826, but not extending into the remaining fluid 816 in the container 804. However, in some cases, the gas supply tubing 820 may extend into the fluid. For example, the opening 826 may be at a bottom or side of the container 804 such that the gas supply tubing 820 terminates within the fluid with gas bubbling up through the fluid 816 to pressurize the container 804. A lumen extends through the gas supply tubing 820 for receiving a flow of air and/or gas therethrough. The lumen of the gas supply tubing 820 is in operative fluid communication with a top portion of the container 804. The opening 826 in the container 804 may be a port configured to receive a spike port adaptor or may be configured to otherwise couple (e.g., a threaded engagement, a friction fit, a snap fit, etc.) with the gas supply tubing 820. In some embodiments, the opening 826 may include a grommet or heat seal configured to seal the container 804 about the tubing 820 in a fluid and pressure tight manner. If so provided, the alternative gas supply tubing may include a pressure relief valve, such as, but not limited to, a 3-way stopcock, a clamp, a spring-loaded valve, or the like to vent pressure within the container 804 and/or to block a flow of pressurized gas to the container 804 during refilling of the container 804, during procedure change-overs, and/or during equipment change-overs.

The lens wash supply tubing 822 extends from a second end external to the reservoir 802 to a first end coupled to a first port of a 3-way port 828, such as, but not limited to, a 3-way stopcock, a 3-way Luer, or the like. The lens wash supply tubing 822 may be fluidly coupled to the fluid 816 in the container 804 via a fluid supply tube or water pick-up tube 830. The 3-way port 828 may be actuated or turned to selectively couple the fluid supply tube 830 with the lens wash supply tube 822. When the lens wash supply tube 822 is fluidly coupled with the fluid supply tube 830, pressurized water or fluid may be delivered to the lens wash supply line 245a of the endoscope 100 when the gas/water valve 140 is depressed.

The fluid supply tube 830 extends from a second end external to the reservoir 802 and coupled to a second port of the 3-way port 828 through a reservoir opening 832, and terminating in a first end within the remaining fluid 816. In some embodiments, the first end of the fluid supply tube 830 may include a floating bobber 834. The floating bobber 834 may maintain the first end of the fluid supply tube 830 within the fluid 816 without requiring the first end of the fluid supply tube 830 to extend towards a bottom portion of the container 804. It is further contemplated that the floating bobber 834 may help negate any impact of hydrostatic pressure on the output of air or water flow. For example, the system 800 may not include any pressure regulation mechanisms downstream of the container 804. Thus, any losses or increases in pressure downstream of the input source (e.g., the first end of the fluid supply tube 830) may lead to potentially inconsistent flow rates over time. In some embodiments, the fluid supply tube 830 may terminate at the opening 832. For example, when the opening 832 is at or adjacent to the bottom portion of the container 804 a dip tube may not be required. A lumen extends through the fluid supply tube 830 for receiving a flow of fluid therethrough. The lumen of the fluid supply tube 830 is in selective operative fluid communication with the fluid 816 of the container 804. In the illustrated embodiment, the gas supply tubing 820 and the fluid supply tube 830 may enter the container 804 through separate openings 826, 832. However, this is not required. The opening 832 in the container 804 may be a port configured to receive a spike port adaptor or may be configured to otherwise couple (e.g., a threaded engagement, a friction fit, a snap fit, etc.) with the fluid supply tube 830. In some embodiments, the opening 832 may include a grommet or heat seal configured to seal the container 804 about the tubing 830 in a fluid and pressure tight manner.

The second ends of the gas supply tubing 820 and the lens wash supply tubing 822 may be connected in fluid communication with the endoscope at gas/lens wash connection 290 on the connector portion 265 of the umbilical. The gas supply tubing 820 is connected in fluid communication with a gas pump (not explicitly shown) and gas feed line (not explicitly shown), and the lens wash supply tubing 822 is connected in fluid communication with lens wash feed line (not explicitly shown), within connector portion 265.

In some embodiments, the reservoir 802 may be connected in fluid communication with a lumen of an upstream irrigation supply tube 824. The upstream irrigation supply tubing 824 extends from a second end region 836 to a first end coupled to a third port of the 3-way port 828. The upstream irrigation supply tube 824 may be fluidly coupled to the fluid 816 in the container 804 via a fluid supply tube or water pick-up tube 830. When irrigation fluid is desired, the 3-way port 828 may be actuated or turned to divert a flow of water or fluid to the upstream irrigation supply tube 824 and the irrigation pump 315.

The second end region 836 of the upstream irrigation supply tube 824 may be disposed within a pump head 324 of the peristaltic irrigation pump 315. The second end of the upstream irrigation supply tube 824 is configured to be fluidly coupled with an irrigation lumen of the endoscope 100. When irrigation water is required, the upstream irrigation supply tube 824 is fluidly coupled with the fluid supply tube 830 and fluid is pumped from the first container 804 by operating the irrigation pump 315, such as by depressing a footswitch (not shown). Water or fluid flows from the first reservoir 802, through the fluid supply tube 830, through the 3-way port 828, through the upstream irrigation supply tubing 824, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 326 positioned in-line with the downstream irrigation supply tubing 255c. The flow control valve 326 may prevent the unintentional flow of fluid from the first container 304 to the endoscope 100. In some cases, the flow control valve 326 may be configured to open when the pressure within the downstream irrigation supply line 255c reaches a predetermined minimum pressure. It is contemplated that the predetermined minimum pressure may be greater than the head pressure created by the height differential between the first reservoir 802 and the irrigation pump 315. The flow control valve 326 may also prevent fluid from leaking from the downstream irrigation supply tube 255c when the endoscope 100 is changed between patients.

It is contemplated that the reservoir 802 may be filled and refilled as needed by fluidly coupling the port 818 with a fluid source, as described above. In some examples, the container 804 may be depressurized prior to filling with water or fluid. The refilling of the reservoir 802 may be performed during a procedure or between procedures, as necessary. Refilling the reservoir 802 via the port 818 may also remove the need to disconnect the reservoir 802 from the tubing 820, 822, 824 throughout the day eliminating or greatly reducing the possibility of cross contamination by removing the need to replace the water reservoir 802.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container and tube set arranged and configured to couple to an endoscope for use in an endoscopic procedure, the container and tube set comprising:
a first container configured to contain a fluid, the first container having a first port in fluid communication with a bottom portion thereof;
a second container configured to contain a fluid, the second container having a first fluid inlet including a first end, a second end, and a first lumen, wherein the first end of the first fluid inlet is coupled to the first port of the first container, the second end of the first fluid inlet is coupled to the second container and the first lumen is in selective fluid communication with the first container;
a first water supply tube including a first end, a second end, and a second lumen extending therethrough, wherein the second lumen is in selective fluid communication with the bottom portion of the second container and the second end of the first water supply tube is positioned external to the second container; and
a first gas supply tube including a first end, a second end, and a third lumen extending therethrough, wherein the third lumen is in operative fluid communication with the second container and the second end of the first gas supply tube is positioned external to the second container.

2. The container and tube set of claim 1, further comprising a second water supply tube including a first end, a second end, and a fourth lumen extending therethrough, wherein the fourth lumen is in selective fluid communication with the bottom portion of the second container and the second end of the second water supply tube is positioned external to the second container.

3. The container and tube set of claim 1, further comprising a second water supply tube including a first end, a second end, and a fourth lumen extending therethrough, wherein the fourth lumen is in selective fluid communication with the bottom portion of the first container and the second end of the second water supply tube is positioned external to the first container.

4. The container and tube set of claim 1, wherein the second container further comprises a partition extending from the bottom portion towards the top portion.

5. The container and tube set of claim 4, wherein the partition divides the second container into a first chamber and a second chamber.

6. The container and tube set of claim 4, wherein the partition terminates a distance from the top portion of the second container.

7. The container and tube set of claim 4, wherein the partition is laterally disposed between a second port of the second container and a third port of the second container.

8. The container and tube set of claim 7, wherein the first end of the first water supply tube is coupled to the second port of the second container and the first end of the first gas supply tube is coupled to the third port of the second container.

9. The container and tube set of claim 4, wherein the partition extends to the top portion of the second container.

10. The container and tube set of claim 9, further comprising a one-way valve disposed within the partition.

11. The container and tube set of claim 1, further comprising a third container configured to contain a fluid, the third container having a second port in fluid communication with a bottom portion thereof.

12. The container and tube set of claim 11, further comprising a second water supply tube including a first end, a second end, and a fourth lumen extending therethrough, wherein the fourth lumen is in selective fluid communication with the bottom portion of the third container and the second end of the second water supply tube is positioned external to the third container.

13. The container and tube set of claim 1, wherein the first container comprises a collapsible bag.

14. The container and tube set of claim 1, wherein the second container comprises a collapsible bag.

15. The container and tube set of claim 1, further comprising a one-way valve positioned between the first end and the second end of the first fluid inlet.

16. A container and tube set arranged and configured to couple to an endoscope for use in an endoscopic procedure, the container and tube set comprising:
a first container configured to contain a fluid, the first container having a first port in fluid communication with a top portion thereof;
a first gas supply tube including a first end, a second end, and a first lumen extending therethrough, wherein the first lumen is in operative fluid communication with the first container and the second end of the first gas supply tube is positioned external to the first container;
a first water supply tube including a first end, a second end, and a second lumen extending therethrough, wherein the second lumen is in selective fluid communication with the fluid of the first container and the second end of the first water supply tube is positioned external to the second container; and
a three-way port coupled the second end of the first water supply tube.

17. The container and tube set of claim 16, further comprising a second water supply tube including a first end, a second end, and a third lumen extending therethrough, wherein the first end is coupled to the three-way port, the third lumen is in selective fluid communication with the first water supply tube and the second end of the second water supply tube is positioned external to the first container.

18. The container and tube set of claim 17, further comprising a third water supply tube including a first end, a second end, and a fourth lumen extending therethrough, wherein the first end is coupled to the three-way port, the fourth lumen is in selective fluid communication with the first water supply tube and the second end of the third water supply tube is positioned external to the first container.

19. The container and tube set of claim 16, wherein the first port is configured to be selectively fluidly coupled with a fluid source.

20. A container and tube set arranged and configured to couple to an endoscope for use in an endoscopic procedure, the container and tube set comprising:
a first container configured to contain a fluid, the first container having a first port in fluid communication with a top portion thereof;
a partition extending from a top portion of the first container to a bottom portion of the first container, the partition dividing the first container into a first chamber and a second chamber;
a one-way valve positioned in the partition, the one-way valve configured to allow a flow of fluid from the first chamber to the second chamber;
a first water supply tube including a first end, a second end, and a first lumen extending therethrough, wherein the first lumen is in selective fluid communication with first chamber of the first container and the second end of the first water supply tube is positioned external to the first container;
a second water supply tube including a first end, a second end, and a second lumen extending therethrough, wherein the second lumen is in selective fluid communication with the bottom portion of the second chamber and the second end of the first water supply tube is positioned external to the first container; and
a first gas supply tube including a first end, a second end, and a third lumen extending therethrough, wherein the third lumen is in operative fluid communication with the second chamber and the second end of the first gas supply tube is positioned external to the first container.
